(19) United States
(12) Patent Application Publication
(10) Pub. No.: US 2012/0028284 A1
(43) Pub. Date: Feb. 2, 2012

(54) TRAPPING COMPOUNDS AND METHOD FOR IDENTIFYING REACTIVE METABOLITES

(75) Inventors: Raku Shinkyo, Tsukuba (JP); Tomomi Ishida, Tsukuba (JP)

(73) Assignee: EISAI R&D MANAGEMENT CO., LTD., Tokyo (JP)

(21) Appl. No.: 13/263,423

(22) PCT Filed: Apr. 16, 2010
(86) PCT No.: PCT/JP2010/057192

§ 371 Date: Oct. 7, 2011

(30) Foreign Application Priority Data
Apr. 23, 2009 (JP) PCT/JP2009/058530

(51) Int. Cl. C12Q 1/48; C12Q 1/26; C07C 321/04

(52) U.S. Cl. 435/15; 562/556; 435/25

(57) ABSTRACT

A compound of formula (I) is useful to identify reactive metabolites.

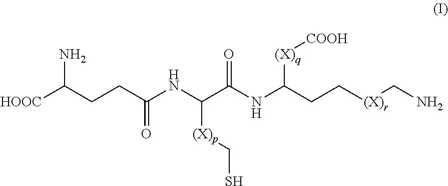

[X is CH₂, and p, q and r are each independently 0 or 1 with a proviso that the compound wherein p is 0, q is 0 and r is 1 is excluded]

### TECHNICAL FIELD

The present invention is directed to non-isotopic trapping compounds, methods for detecting reactive metabolites and methods of identifying drug candidates. More specifically, the non-isotopic trapping compounds and methods for detecting reactive metabolites may be utilized to detect “soft” and “hard” reactive metabolites and furthermore, acylglucuronides.

### BACKGROUND ART

In a case of patient morbidity and mortality, particular toxicity of the compounds remains a serious safety concern in both clinical development and after market launch. These particular drug adverse events can lead to restrictive use and even stand-down from the market, which consequently results in higher burden for the pharmaceutical companies. As a famous instance, troglitazone was retreated immediately after its launch because of unexpected toxicity.

Particular drug adverse events are a rare case which ordinarily shows in a high degree of individual susceptibility. Furthermore, these reactions are commonly occurred in a dose-independent manner. At present, there are no animal models that can be used to evaluate such reactions that exclusively occur in humans. Therefore, the particular drug toxicities cannot be correctly evaluated in preclinical studies, and are often unrecognizable in clinical trials.

At present time, the mechanisms of the particular adverse drug reactions are not well understood. A large number of evidence to suggest that chemically reactive metabolites are involved in particular toxicities, especially for liver toxicity. Many compounds related to particular toxicity produce reactive metabolites through various metabolic pathways mediated predominately by cytochrome P450 enzymes (CYPs), similarly by other oxidative enzymes such as peroxidases, cyclooxygenases and myeloperoxidases or through another metabolic pathway mediated by UDP-glucuronosyltransferases (UGTs). It presumes many compounds related such toxicities first undergo metabolic activation to produce toxic reactive metabolites that covalently bind to cellular proteins. These covalently modified proteins are often antigenic and thus stimulate an immune response, resulting in particular drug reactions. An another hypothesis states that covalent modifications of cellular proteins by reactive metabolites block signal transduction cascades and essential functions of cells, leading to fatal consequences observed in clinic. Consequently there is a serious need for methods for identifying reactive metabolites.

Reactive metabolites of chemical compounds produced by oxidative enzymes can be characterized into two categories based on their chemical properties as “soft” and “hard” reactive metabolites. “Soft” reactive metabolites comprise a majority of electrophilic metabolites which include quinones, quinone imines, iminoquinone methides, epoxides, arene oxides and nitrenium ions, and easily react with “soft” nucleophiles such as the sulfhydryl group in cysteine. In contrast, “hard” reactive metabolites, most commonly seen as aldehydes, preferentially react to “hard” nucleophiles such as amines of lysine, arginine and nucleic acids. Because of their instability, direct detection and characterization of reactive metabolites has proven to be extremely difficult. A commonly utilized approach is to trap these reactive metabolites with a capture molecule, resulting in formation of a stable adduct that can be subsequently characterized by known detection methods, for example by tandem mass spectrometry.

Another category of reactive metabolites produced by UGTs are acylglucuronides. Acylglucuronides are intrinsically reactive species and undergo intramolecular rearrangement and anomerization followed by a reaction with proteins to produce covalent adducts. At present, there is no report about the detection system of acylglucuronides with trapping agent using neutral loss tandem mass spectrometry.

Recently, Huebert et al., in WO 2008/042634 A2 disclosed a method for detecting reactive metabolites using stable isotope-labeled trapping compounds and mass spectrometry. However, Huebert et al. merely disclosed a method using “stable isotope-labeled compounds” but did not show any “non-isotopically labeled compounds” as an exemplified embodiment. Furthermore, the method as disclosed by Huebert et al., can detect only “soft” and “hard” reactive metabolites, but cannot detect any “acylglucuronides” as an exemplified one.

On the other hand, the present invention indicates the particular feature in which a non-isotopic compound of formula (I) can endow the new method for the detection “soft” and “hard” reactive metabolites and further, acylglucuronides in a single experiment.

### SUMMARY OF INVENTION

The present invention is directed to a non-isotopic trapping compound for identifying reactive metabolites (including “soft” and “hard” reactive metabolites and acylglucuronides), a compound of formula (I):

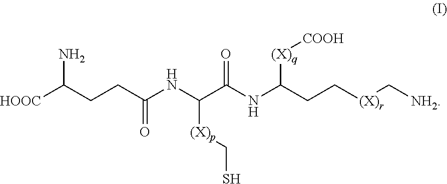

The present invention is further directed to a method for detecting reactive metabolites of a test compound comprising

(a) incubating a test compound with the compound of formula (I) above and a drug metabolizing enzyme; to yield a product mixture comprising one or more adducts; and

(b) detecting the adducts of Step (a).

### BRIEF DESCRIPTION OF DRAWINGS

FIG. 1A illustrates the total ion chromatogram (TIC; upper) and TIC of neutral loss scanning of 129 Da (middle) and 147 Da (lower) for diclofenac when analyzed with ECO.

FIG. 1B illustrates the full scan (upper) and product ion spectra (lower) of adduct I derived from diclofenac when analyzed with ECO.

FIG. 1C illustrates the full scan (upper) and product ion spectra (lower) of adduct II derived from diclofenac when analyzed with ECO.

FIG. 2A illustrates the TIC (upper) and TIC of neutral loss scanning of 129 Da (middle) and 147 Da (lower) for clozapine when analyzed with ECO.

FIG. 2B illustrates the full scan (upper) and product ion spectra (lower) of adduct I derived from clozapine when analyzed with ECO.

FIG. 3A illustrates the TIC (upper) and TIC of neutral loss scanning of 129 Da (middle) and 147 Da (lower) for 2-[2-thienyl]-furan when analyzed with ECO.

FIG. 3B illustrates the full scan (upper) and product ion spectra (lower) of adduct I derived from 2-[2-thienyl]-furan when analyzed with ECO.

FIG. 3C illustrates the full scan (upper) and product ion spectra (lower) of adduct II derived from 2-[2-thienyl]-furan when analyzed with ECO.

FIG. 3D illustrates the full scan (upper) and product ion spectra (lower) of adduct III derived from 2-[2-thienyl]-furan when analyzed with ECO.

FIG. 4A illustrates the TIC (upper) and TIC of neutral loss scanning of 129 Da (middle) and 147 Da (lower) for diclofenac acylglucuronide when analyzed with ECO.

FIG. 4B illustrates the full scan (upper) and product ion spectra (lower) of adduct I derived from diclofenac acylglucuronide when analyzed with ECO.

FIG. 5A illustrates the TIC (upper) and TIC of neutral loss scanning of 129 Da (lower) for diclofenac when analyzed with ECHK.

FIG. 5B illustrates the full scan (upper) and product ion spectra (lower) of adduct I derived from diclofenac when analyzed with ECHK.

FIG. 5C illustrates the full scan (upper) and product ion spectra (lower) of adduct II derived from diclofenac when analyzed with ECHK.

FIG. 6A illustrates the TIC (upper) and TIC of neutral loss scanning of 129 Da (lower) for clozapine when analyzed with ECHK.

FIG. 6B illustrates the full scan (upper) and product ion spectra (lower) of adduct I derived from clozapine when analyzed with ECHK.

FIG. 7A illustrates the TIC (upper) and TIC of neutral loss scanning of 129 Da (lower) for 2-[2-thienyl]-furan when analyzed with ECHK.

FIG. 7B illustrates the full scan (upper) and product ion spectra (lower) of adduct I derived from 2-[2-thienyl]-furan when analyzed with ECHK.

FIG. 7C illustrates the full scan (upper) and product ion spectra (lower) of adduct II derived from 2-[2-thienyl]-furan when analyzed with ECHK.

FIG. 7D illustrates the full scan (upper) and product ion spectra (lower) of adduct III derived from 2-[2-thienyl]-furan when analyzed with ECHK.

FIG. 8A illustrates the TIC (upper) and TIC of neutral loss scanning of 129 Da (lower) for diclofenac acylglucuronide when analyzed with ECHK.

FIG. 8B illustrates the full scan (upper) and product ion spectra (lower) of adduct I derived from diclofenac acylglucuronide when analyzed with ECHK.

FIG. 9A illustrates the TIC (upper) and TIC of neutral loss scanning of 129 Da (lower) for diclofenac when analyzed with EHCK.

FIG. 9B illustrates the full scan (upper) and product ion spectra (lower) of adduct I derived from diclofenac when analyzed with EHCK.

FIG. 9C illustrates the full scan (upper) and product ion spectra (lower) of adduct II derived from diclofenac when analyzed with EHCK.

FIG. 10A illustrates the TIC (upper) and TIC of neutral loss scanning of 129 Da (lower) for clozapine when analyzed with EHCK.

FIG. 10B illustrates the full scan (upper) and product ion spectra (lower) of adduct I derived from clozapine when analyzed with EHCK.

FIG. 11A illustrates the TIC (upper) and TIC of neutral loss scanning of 129 Da (lower) for 2-[2-thienyl]-furan when analyzed with EHCK.

FIG. 11B illustrates the full scan (upper) and product ion spectra (lower) of adduct I derived from 2-[2-thienyl]-furan when analyzed with EHCK.

FIG. 11C illustrates the full scan (upper) and product ion spectra (lower) of adduct II derived from 2-[2-thienyl]-furan when analyzed with EHCK.

FIG. 11D illustrates the full scan (upper) and product ion spectra (lower) of adduct III derived from 2[2-thienyl]-furan when analyzed with EHCK.

FIG. 12A illustrates the TIC (upper) and TIC of neutral loss scanning of 129 Da (lower) for diclofenac acylglucuronide when analyzed with EHCK.

FIG. 12B illustrates the full scan (upper) and product ion spectra (lower) of adduct I derived from diclofenac acylglucuronide when analyzed with EHCK.

### DESCRIPTION OF EMBODIMENTS

The present invention is directed to a non-isotopic trapping compound, wherein the trapping compound is capable of binding to reactive metabolites, a compound of formula (I):

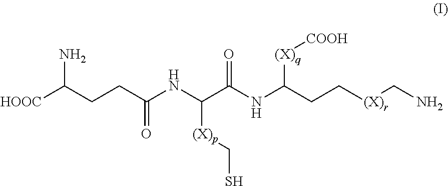

wherein

X is CH₂, and

p, q and r are each independently 0 or 1

with a proviso that the compound wherein p is 0, q is 0 and r is 1 is excluded.

The compound of formula (I) is a tripeptide derivative. The compound of formula (I) can be prepared by solid-phase peptide synthesis from protected amino acids (glutamic acid, cysteine/homocysteine and lysine/homolysine/ornithine/β-lysine).

The compound of formula (I) is preferably the compound of formula (II), (III) or (IV):

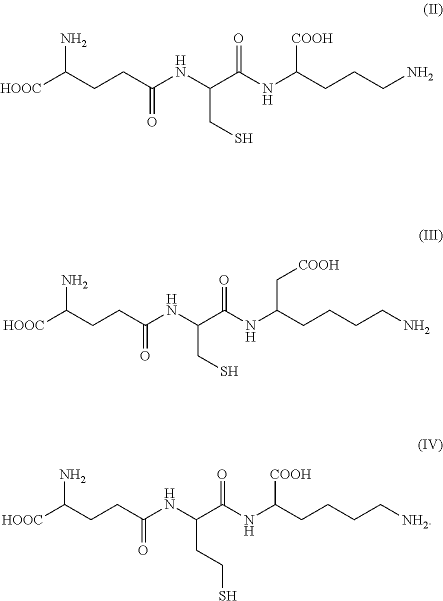

The compound of formula (II) is γ-glutamylcysteinylornithine abbreviated as ECO, the compound of formula (III) is γ-glutamylcysteinylhomolysine abbreviated as ECHK, and the compound of formula (IV) is γ-glutamylhomocysteinyllysine abbreviated as EHCK.

The compounds of formulae (I) to (IV) have three chiral carbons and there exist various stereoisomers. The compounds of formulae (I) to (IV) may be any stereoisomer or mixture thereof. Preferably the compounds of formulae (I) to (IV) substantially include a certain stereoisomer only. More preferably, the amino acids which make up the compounds of formulae (I) to (IV) are all in L-form or all in D-form.

The compound of formula (I) has two “trapping zones”, which individually trap “hard” and “soft” reactive metabolites. More specifically, the sulfhydryl (—SH) group on the compound of formula (I) reacts with and traps so-called “soft” reactive metabolites, whereas the —(X)ᵣ—CH₂—NH₂ group on the compound of formula (I) reacts with and traps so-called “hard” reactive metabolites. Thus the compound of formula (I) is capable of simultaneously trapping both “hard” and “soft” reactive metabolites.

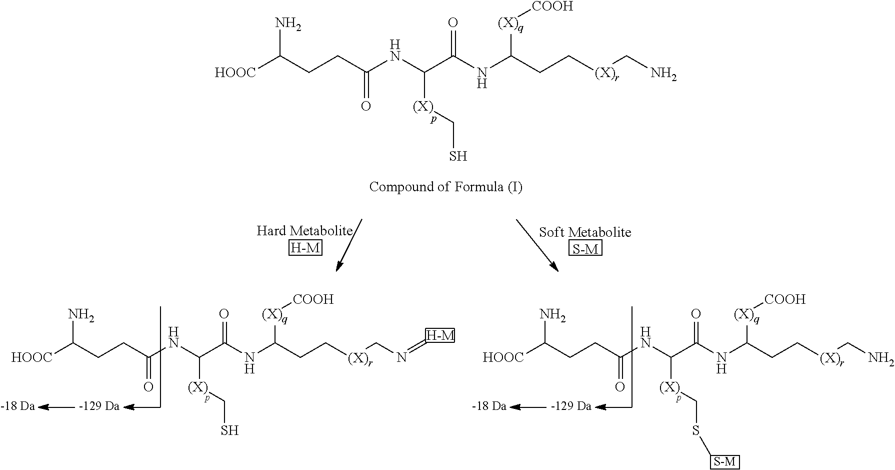

As used herein, unless otherwise noted, the term “reactive metabolites” shall include “soft” and “hard” reactive metabolites and acylglucuronides.

As used herein, unless otherwise noted, the term “soft metabolite” shall mean any electrophilic metabolite which comprises at least one substituent group which readily reacts with “soft” nucleophiles such as the sulfhydryl group in cysteine and the —SH group on the compound of formula (I). Suitable examples of such substituent groups include, but are not limited to quinones, quinone imines, iminoquinone, methids, epoxides, arene oxides, nitrenium ions, and the like.

As used herein, unless otherwise noted, the term “hard metabolite” shall mean any electrophilic metabolite which comprises at least one substituent group which readily reacts with “hard” nucleophiles such as the amines of lysine, arginine or the —(X)ᵣ—CH₂-NH₂ group of the compound of formula (I). Suitable examples of such substituent groups include, but are not limited to aldehydes, and the like.

As used herein, unless otherwise noted, the taut “acylglucuronide” shall mean any reactive metabolite which reacts with nucleophiles such as the sulfhydryl group in cysteine, —SH group of the compound of formula (I), the amines of lysine, arginine or the —(X)ᵣ—CH₂—NH₂ group of the compound of formula (I). Suitable examples of such substituent groups include, but are not limited to 1-O-β-acyleucuronide, and the like.

Since reactive metabolites are implicated in many adverse events associated with test compounds, particularly serious and/or toxic adverse events, it is highly desirable to be able to detect all reactive metabolites produced by a test compound. It is further highly desirable to determine whether a test compound will produce reactive metabolites prior to administration of the test compound to humans.

One skilled in the art will recognize that not all test compounds produce reactive metabolites and further, that not all test compounds produce “soft” and “hard” reactive metabolites and acylglucuronides. Some test compounds will produce no reactive metabolites, some test compounds will produce only “hard” reactive metabolites, some test compounds will produce only “soft” reactive metabolites, some test compounds will produce only acylglucuronides, and some test compounds will produce “soft” and “hard” reactive metabolites, and acylglucuronides. Thus the trapping compounds and methods of the present invention will detect any type of reactive metabolite that is produced by a test compound.

One skilled in the art will further recognize that the methods of the present invention, while directed to identifying reactive metabolites, also encompass the process of determining whether or not a test compound produces reactive metabolites. In an embodiment, the present invention is directed to a method for identifying hopeful drug candidates (e.g. test compounds which do not produce reactive metabolites). Thus, the methods of the present invention include processes wherein the incubation (as described in more detail herein) results in no reactive metabolites and thus no adducts, and the total ion spectra of neutral loss scanning of 129 Da or 147 Da show no peaks, thereby indicating that the test compound does not produce any detectable reactive metabolites.

For “soft” metabolites, glutathione is the most common trapping agent used in microsomal incubations, for detecting “soft” reactive metabolites. However, for “hard” reactive metabolites and acylglucuronides, glutathione is not a suitable trapping agent because of its trapping efficiency. Rather, for detecting “hard” reactive metabolites alternative trapping agents such as semicarbazide, methoxylamine and α-acetyllysine are used. On the other hand, for detecting acylglucuronides, N-acetylcysteine (NAC) is useful. From the reasons above, these require three individual experiments with different trapping agents for the detection of “hard” and “soft” reactive metabolites and acylglucuronides. Even in the situation, the present invention indicates the particular feature in which the non-isotopically labeled compound of formula (I) can endow the new method for the detection “soft” and “hard” reactive metabolites and further, acylglucuronides in a single experiment.

As used herein, unless otherwise noted “drug metabolizing enzyme” shall mean any enzyme or mixture thereof, derived from human or animal tissues, preferably derived from human, rat, mouse, hamster, dog, monkey or rabbit tissues, more preferably derived from human, rat, mouse, hamster, dog, monkey or rabbit liver tissue, which can metabolize a test compound. (See for example, Drug Metabolizing Enzymes, Edited by Jae S. Lee, R. Scott Obach and Michael B. Fisher, Marcel Dekker, inc. (2003)) Suitable examples include, but are not limited to liver microsomes, cytochrome P450 enzymes or mixtures of different isoforms of cytochrome P450 enzymes, peroxidases, cyclooxygenases, myeloperoxidases, UDP-glucuronosyltransferases (UGTs) and the like. Preferably, the drug metabolizing enzymes are human liver microsomes, more preferably, cytochrome P450 enzymes, recombinant enzymes and UGTs. One skilled in the art will recognize that when incubating a test compound with UGTs and cytochrome P450 enzymes or mixtures of different isoforms of cytochrome P450 enzymes, the UGTs and cytochrome P450 enzymes or mixtures of different isoforms of cytochrome P450 are incubated in combination with Uridine diphosphoglucuronic acid (UDPGA) and NADPH co-factor or the NADPH regenerating system.

As used herein, unless otherwise noted, the term “test compound” shall mean any chemical which is tested for the formation of reactive metabolite(s). Preferably, the test compound is a pharmaceutical agent or salt, ester or prodrug thereof.

As used herein, unless otherwise noted, the term “drug candidate” shall mean any chemical or test compound which does not produce reactive metabolite(s). Preferably, the drug candidate is a pharmaceutical agent or salt, ester or prodrug thereof.

As used herein, unless otherwise indicated, the term “adduct” shall mean any covalently bonded complex of a reactive metabolite with a compound of formula (I).

Abbreviations used in the specification, particularly the Schemes and Examples, are as follows:

APCI-MS/MS=Atmospheric pressure chemical ionization tandem mass spectrometry

CYPs=Cytochrome P450 enzymes

UGTs=UDP-glucuronosyltransferases

Da=Daltons

ESI-MS/MS=Electrospray Ionization tandem mass spectrometry

HPLC=High performance liquid chromatography

MS=Mass spectrometry

NADPH=β-nicotinamide adenine dinucleotide phosphate (reduced)

SPE=Solid phase extraction

UDPGA=Uridine diphosphoglucuronic acid

In an embodiment of the present invention, the drug metabolizing enzyme is selected from the group consisting of human liver microsomes, cytochrome P450 enzymes, peroxidases, cyclooxygenases, myeloperoxidases and UGTs. Preferably, the drug metabolizing enzyme is cytochrome P450 enzymes and UGTs.

In an embodiment, the present invention is applied to detecting reactive metabolites formed by incubating a test compound with any fraction of cells containing drug metabolizing enzymes, for example, S9, recombinant enzymes or microsomal enzymes. In an embodiment, the methods of the present invention are applied to predicting whether or not a test compound will form reactive metabolites in human subjects (i.e. following administration of the test compound to a human).

One skilled in the art will understand that the abbreviation “S9” refers to the S9 fraction (post-mitochondrial supernatant fraction) which is a mixture of microsomes and cytosol. Accordingly, it contains a wide variety of phase I and phase II enzymes including P450 enzymes, flavin-monooxygenases, carboxylesterases, epoxide hydrolase, UDP-glucuronosyltransferases, sulfotransferases, methyltransferases, acetyltransferases, glutathione S-transferases and other drug-metabolizing enzyme.

The product mixture containing one or more of the incubation products (non-reactive metabolite(s), adducts formed between reactive metabolite(s) and the compound of formula (I)) is preferably cleaned and concentrated according to known methods, for example by centrifugation, SPE, evaporation or liquid-liquid extractions, to yield a product concentrate. The product concentrate is then dissolved in a solvent suitable for use in mass spectrometry (i.e. suitable for injection into a mass spectrometer), for example, 5% acetonitrile in water, 5% methanol in water, and the like.

Preferably, the product mixture is separated into individual adduct components according to known methods, for example by liquid chromatography, HPLC, capillary electrophoresis, or other separation technique. A neutral loss mass spectrum is then measured for each adduct or adduct component. The neutral loss mass spectrum may be measured according to known methods, using any ionization source, for example by APCI-MS/MS, ESI-MS/MS, and the like, preferably by ESI-MS/MS. Alternatively, the separation and mass spectrum measurement may be completed in one step using a loop system such as, LC/MS, and the like.

The methods of the present invention are intended for determining whether a test compound will produce “hard” and/or “soft” reactive metabolites and/or acylglucuronides, in a single experiment. One skilled in the art will recognize that although the methods of the present invention will simultaneously detect both “hard” and “soft” reactive metabolites and acylglucuronides, if either “hard”, “soft” reactive metabolites or acylglucuronides are not formed from a particular test compound, the method will be conducted to detect whichever type of reactive metabolite is formed. Additionally, if no reactive metabolites are formed, the method will produce a mass spectrum which shows that there are no peaks in the total ion spectra of neutral loss scanning of 129 Da and 147 Da, thereby identifying the test compound as a drug candidate.

The present invention is further directed to a method for identifying a drug candidate (i.e. a test compound which does not produce any reactive metabolite) comprising

(a) incubating a test compound with a mixture comprising a non-isotopically labeled compound of formula (I), and a drug metabolizing enzyme, to yield a product mixture;
(b) measuring a neutral loss mass spectrum of the product mixture produced in step (a); and
(c) detecting the absence of peaks in neutral loss mass spectrum.

The following examples are set forth to aid in the understanding of the invention, and are not intended and should not be construed to limit in anyway the invention set forth in the claims which follow thereafter.

### EXAMPLES

### Example 1

### Standard Procedures for Trapping “Soft” and “Hard” Reactive Metabolites

### A. Incubation & Compound Trapping

All microsomal incubations described herein were performed at 37° C. in a water bath or on a heat block. The test compound was mixed with human liver microsomal preparations (cytochrome P450 enzyme preparations) in 100 mM potassium phosphate buffer (pH7.4) including 0.1 mM EDTA and the compound of formula (I). The resulting mixtures were pre-warmed at 37° C. for 5 min. To the reaction mixtures was then added the co-factor-NADPH (to initiate the reaction) to yield a final volume of 500 μl.

The resulting reaction mixtures contained 50 μM test compounds, 2 mg/ml microsomal proteins, 1 mM mixture of compound of formula (I) and 3 mM NADPH.

After 15 or 60 min incubation, the reactions were terminated by the addition of 1000 μl of 50% methanol/50% acetonitrile. The resulting mixtures were centrifuged at 10,000 g for 10 min at 4° C. to pellet the precipitated protein, and the supernatants were dried up. The resulting residue was solubilized with 50% methanol.

### B. Mass Spectrometry

MS analyses were performed on an LTQ Orbitrap FTMS (Thermo Fisher Scientific, Inc., Bremen, Germany). The ESI ion source was operated in the positive ion mode. The entire eluent was sprayed into the mass spectrometer at +5 kV and desolvation of the solvent droplets was further aided by a heated capillary temperature of 350° C. Mass spectra collected in the neutral loss scanning mode were obtained by scanning over the range m/z 200-1100 at 30000 resolution.

### C. LC-MS/MS Analyses

For complete profiling of reactive metabolites, samples were first subjected to chromatographic separations with a Prominence LC system with an auto-sampler (Shimadzu Corp., Kyoto, Japan) and eluents were introduced to the LTQ Orbitrap FTMS operated in the neutral loss scanning mode. An L-column ODS (Octadecylsilyl-silica gels; 2.1×150 mm, Chemicals Evaluation and Research Institute, Tokyo, Japan) was used for the chromatographic separation. The chromatographic system used a binary solvent system delivered as a gradient of solvent A (5 mM ammonium acetate) and solvent B (95% acetonitrile included 5 mM ammonium acetate). The initial gradient conditions were 95% A:5% B in for 4 min followed by a linear gradient up to 100% B over the next 13 min at a flow rate of 0.25 mL/min. The solvent composition was then held at 100% B for 8 min before re-equilibration at initial conditions. LC-MS/MS analyses were carried out on 20 μl aliquots of cleaned samples. Data acquisition was carried out using the Xcaliber version 2.0 software (Thermo Fisher Scientific, Inc., Bremen, Germany).

### Example 2-4

### Detecting Soft and/or Hard Reactive Metabolites

Following the procedure as described in Example 1 above, the method of the present invention and ECO were applied to detect reactive metabolites of test compounds which are known to produce “soft” and for “hard” reactive metabolites, using neutral loss scanning of 129 Da or 147 Da.

### Example 2

### Diclofenac Analyzed by ECO

Diclofenac was selected as a test compound to evaluate the usefulness of the present method and ECO to detect “soft” reactive metabolites.

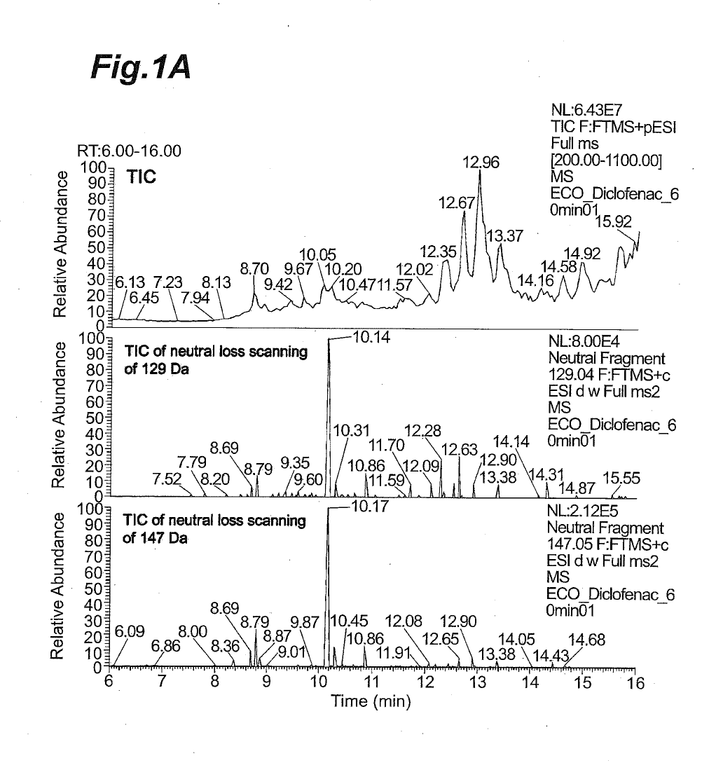

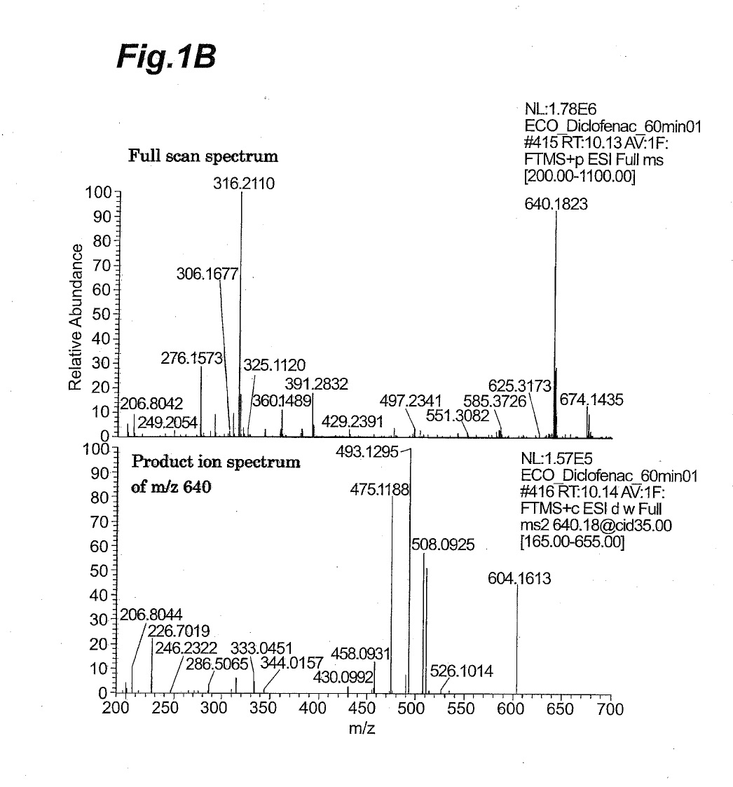

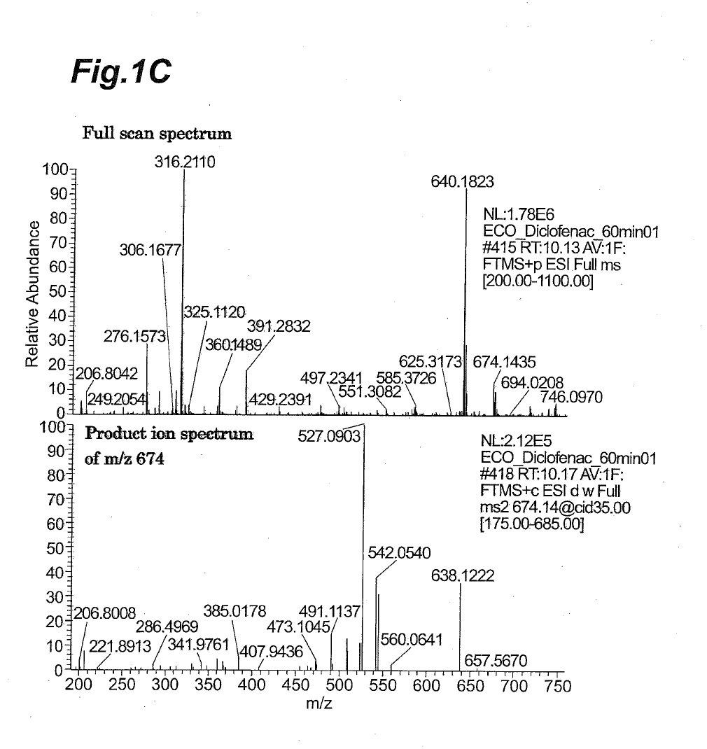

FIG. 1A shows total ion chromatogram (TIC) and TIC of neutral loss scanning of 129 Da and 147 Da obtained for the reaction mixture. The most intense peak which was detected in TIC of neutral loss scanning of 147 Da at retention time of 10.2 min included two adducts of reactive metabolites of diclofenac with ECO which showed the protonated ions at m/z 640 and 674. Full scan and product ion spectra of the two adducts were shown in FIGS. 1B and 1C, respectively.

The formation of the two adducts of reactive metabolites of diclofenac with ECU which were detected in the present method is as outlined in Scheme E1, below.

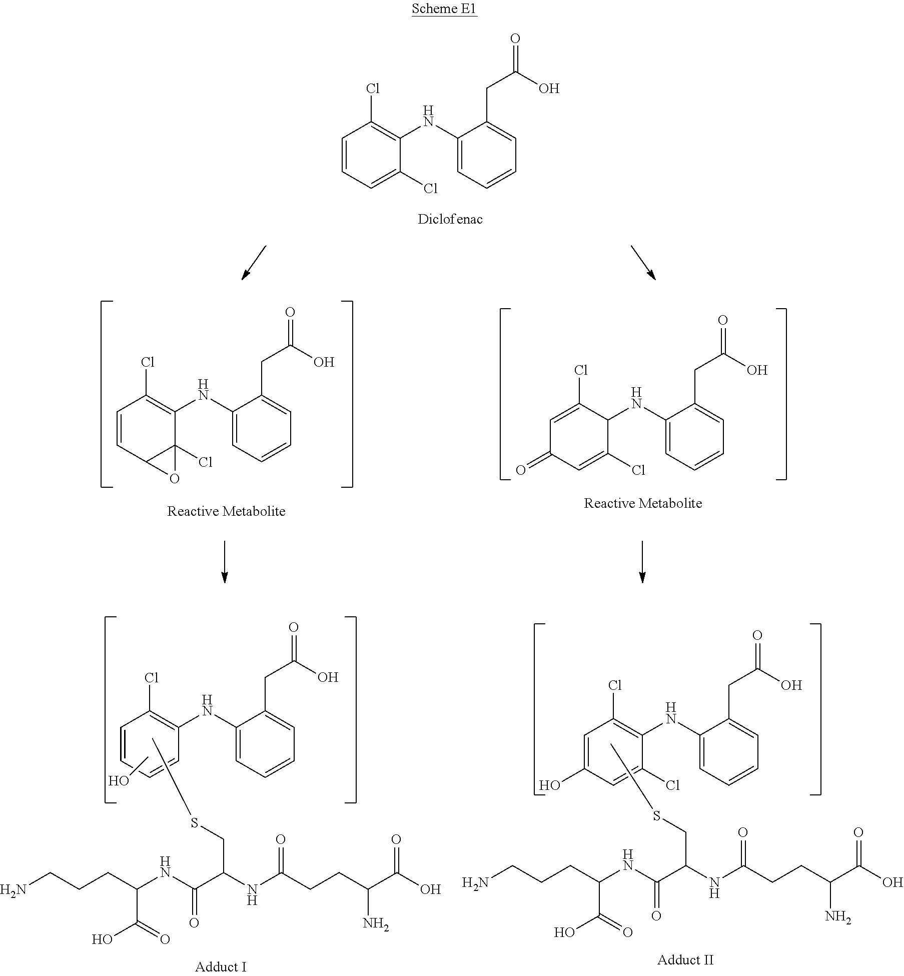

### Example 3

### Clozapine Analyzed by ECO

Clozapine was selected as a test compound to evaluate the usefulness of the present method and ECO to detect a “soft” reactive metabolite.

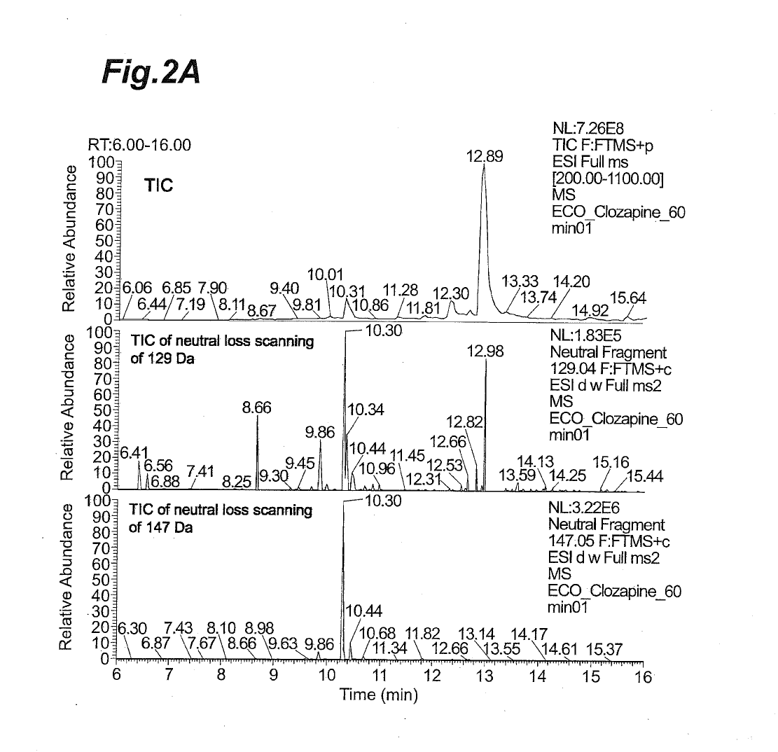

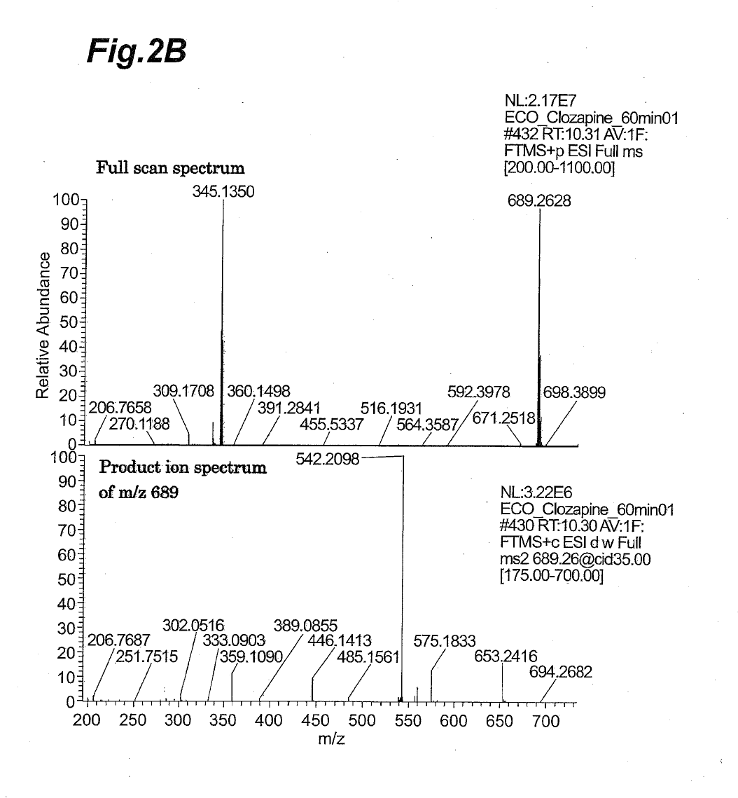

FIG. 2A shows TIC and TIC of neutral loss scanning of 129 Da and 147 Da obtained for the reaction mixture. The most intense peak which was detected in TIC of neutral loss scanning of 147 Da at retention time of 10.3 min included a single adduct which showed the protonated ion at m/z 689. Full scan and product ion spectra of this adduct was shown in FIG. 2B.

The formation of adduct of the reactive metabolite of clozapine with ECO which was detected in the present method is as outlined in Scheme E2, below.

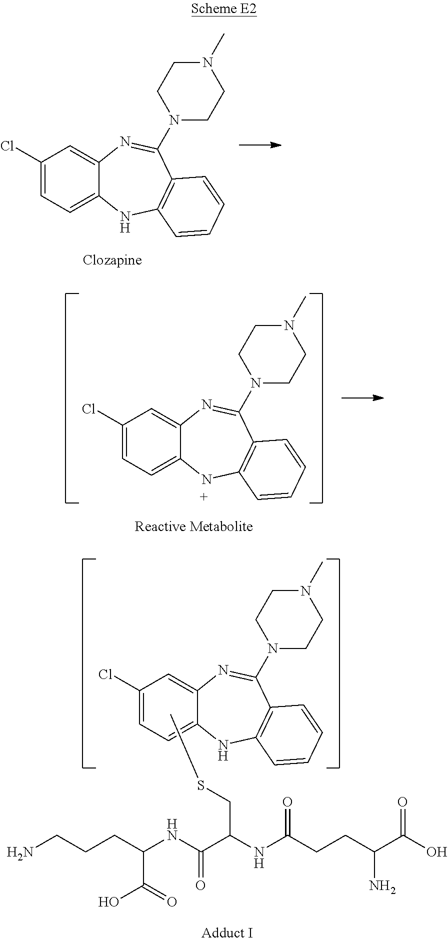

### Example 4

### 2-[2-Thienyl]-furan Analyzed by ECO

2-[2-Thienyl]-furan was selected as a test compound to evaluate the usefulness of the present method and ECO to detect both “soft” and “hard” reactive metabolites.

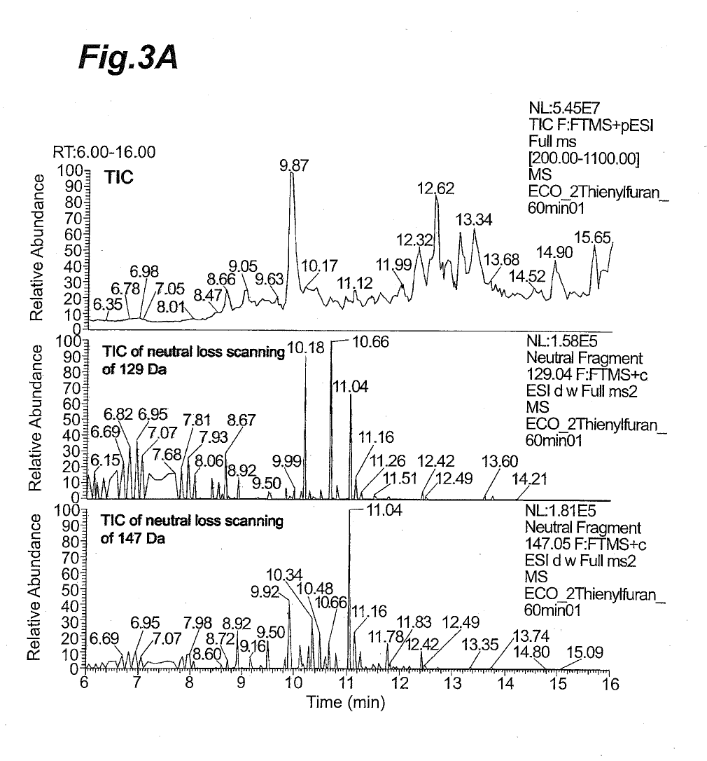

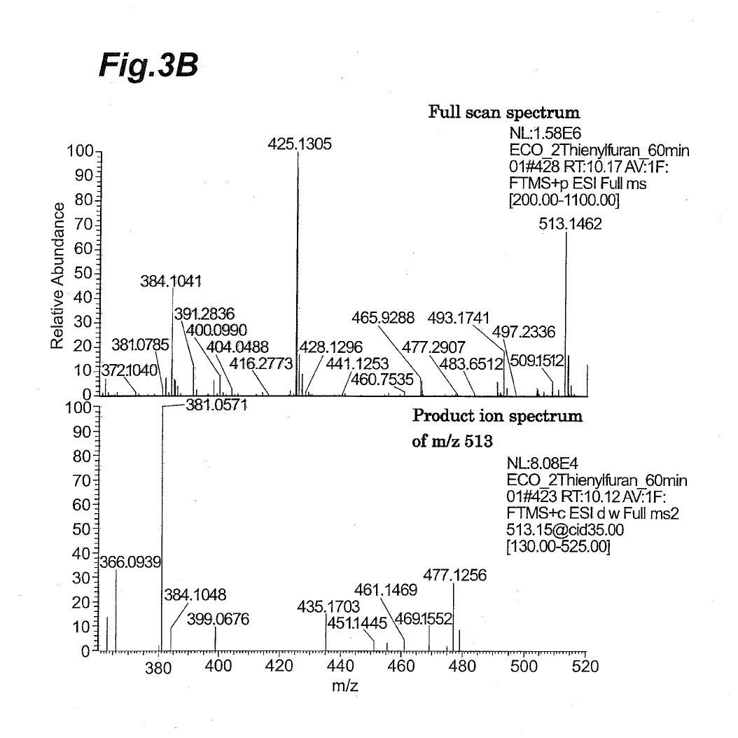

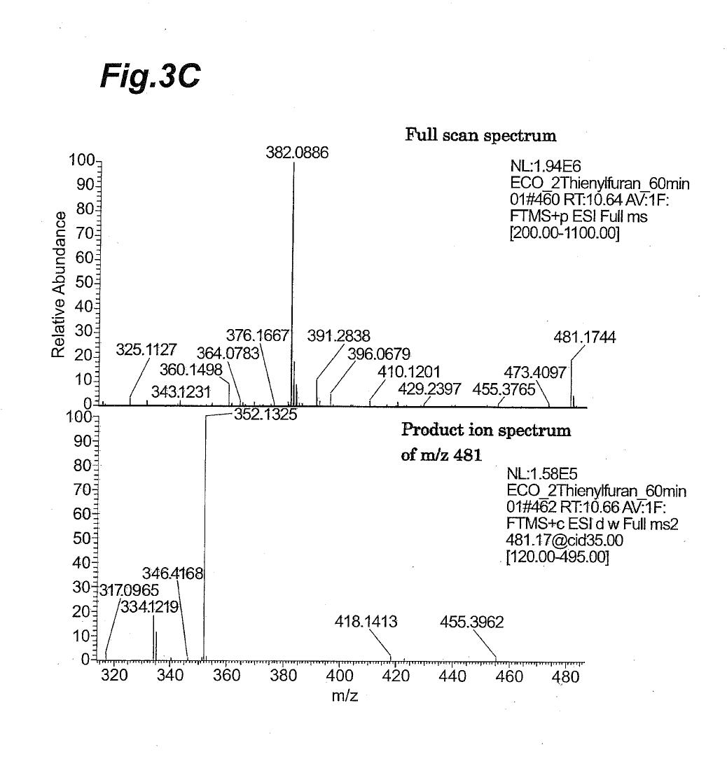

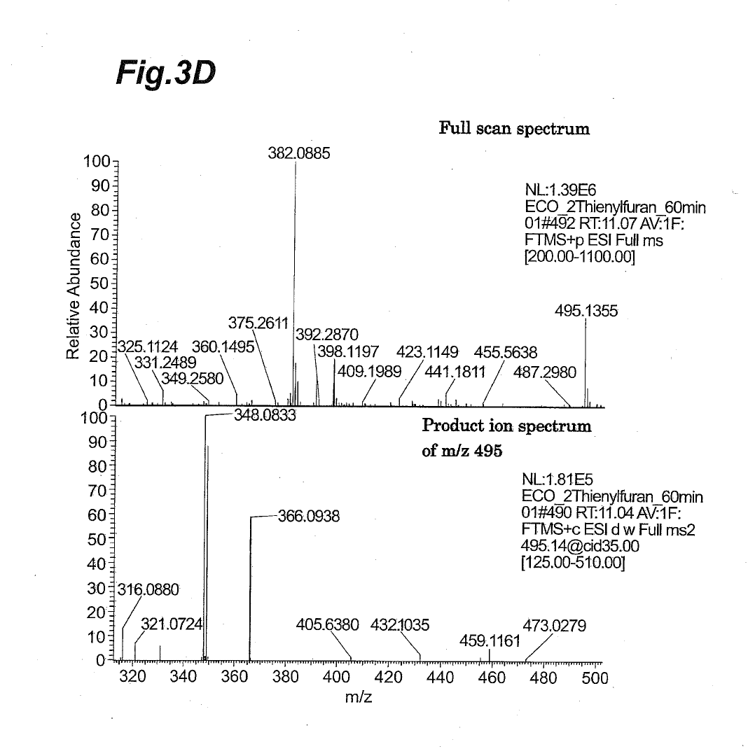

FIG. 3A shows TIC and TIC of neutral loss scanning of 129 Da and 147 Da obtained for the reaction mixture. Three intense peaks were detected in TIC of neutral loss scanning of 129 Da at retention time of 10.2 min, 10.7 min and 11.0 min. The first component at 10.2 min showed the protonated ion at m/z 513 in full scan spectrum and the product ion at m/z 384 which indicated the loss of pyroglutamate moiety in product ion spectrum, as shown in FIG. 3B. The second component at 10.7 min showed the protonated ion at m/z 481 in full scan spectrum and the product ion at m/z 352 which indicated the loss of pyroglutamate moiety in product ion spectrum, as shown in FIG. 3C. The third component at 11.0 min showed the protonated ion at m/z 495 in full scan spectrum and the product ion at m/z 366 which indicated the loss of pyroglutamate moiety in product ion spectrum, as shown in FIG. 3D.

The formation of the three adducts of reactive metabolites of 2-[2-thienyl]-furan with ECO which were detected in the present method is as outlined in Scheme E3, below.

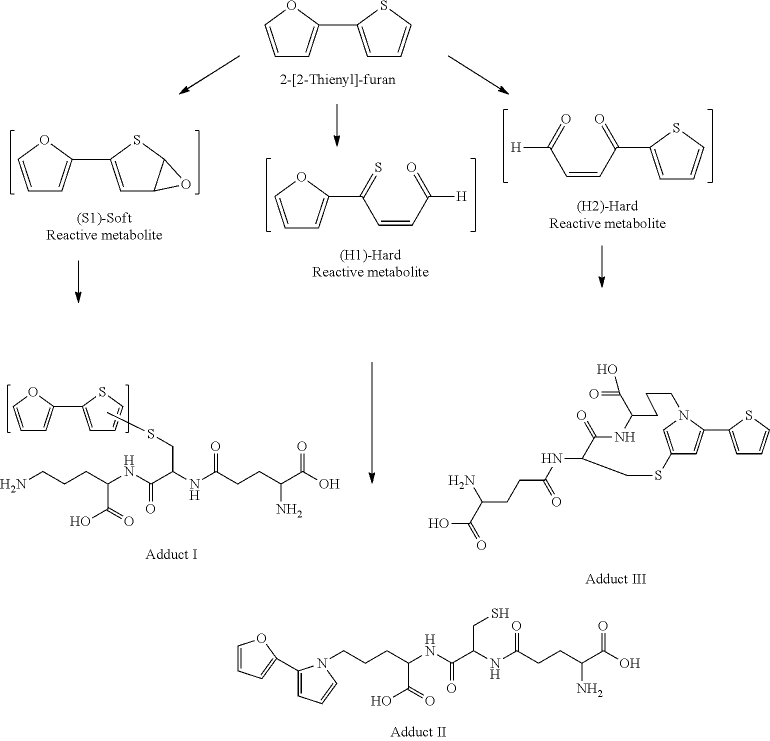

### Example 5

### Standard Procedures for Trapping Acylglucuronides

### A. Incubation & Compound Trapping

All incubations described herein were performed at 37° C. in a water bath or on a heat block. Diclofenac acylglucuronide was added in 100 mM potassium phosphate buffer (pH7.4) including 0.1 mM EDTA. The resulting mixtures were pre-warmed at 37° C. for 5 min. To the reaction mixtures was then added to the compound of formula (I) (to initiate the reaction) to yield a final volume of 500 d.

The resulting reaction mixtures contained 100 μM diclofenac acylglucuronide, 1 mM mixture of compound of formula (I).

After 60 min incubation, the incubations were stopped by placing on ice and 20-μl aliquots of samples were applied to LC/MS/MS analyses.

### B. Mass Spectrometry

MS analyses were performed on an LTQ Orbitrap FTMS (Thermo Fisher Scientific, Inc., Bremen, Germany). The ESI ion source was operated in the positive ion mode. The entire eluent was sprayed into the mass spectrometer at +5 kV and desolvation of the solvent droplets was further aided by a heated capillary temperature of 350° C. Mass spectra collected in the neutral loss scanning mode were obtained by scanning over the range m/z 200-1100 at 30000 resolution.

### C. LC-MS/MS Analyses

For complete profiling of reactive metabolites, samples were first subjected to chromatographic separations with a Prominence LC system with an auto-sampler (Shimadzu Corp., Kyoto, Japan) and eluents were introduced to the LTQ Orbitrap FTMS operated in the neutral loss scanning mode. An L-column ODS (2.1×150 mm, Chemicals Evaluation and Research Institute, Tokyo, Japan) was used for the chromatographic separation. The chromatographic system used a binary solvent system delivered as a gradient of solvent A (5 mM ammonium acetate) and solvent B (95% acetonitrile including 5 mM ammonium acetate). The initial gradient conditions were 95% A:5% B for 4 min followed by a linear gradient up to 100% B over the next 13 min. The solvent composition was then held at 100% B for 8 min before re-equilibration at initial conditions. LC-MS/MS analyses were carried out on 20-μl aliquots of cleaned samples. Data acquisition was carried out using the Xcaliber version 2.0 software (Thermo Fisher Scientific, Inc., Bremen, Germany).

### Example 6

### Diclofenac Acylglucuronide Analyzed by ECO

Diclofenac acylglucuronide was selected as a test compound to evaluate the usefulness of the present method and ECO to detect reactive metabolites which were produced by UDP-glucuronosyltransferase.

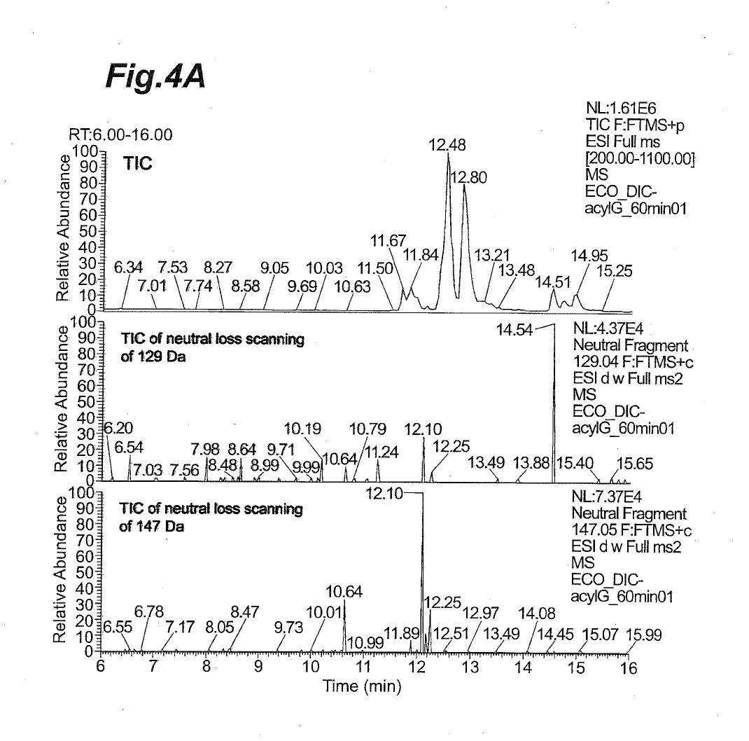

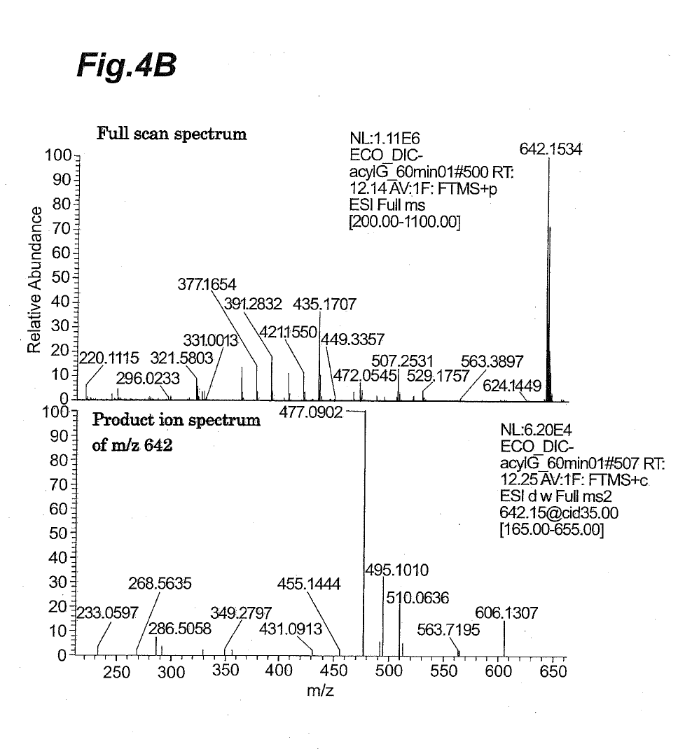

FIG. 4A shows TIC and TIC of neutral loss scanning of 129 Da and 147 Da obtained for the reaction mixture. The common intense peak was detected in TIC of neutral loss scanning of 129 Da and 147 Da at retention time of 12.1 min. This peak shows the protonated ion at m/z 642 in full scan spectrum and the product ion at m/z 495 which indicated the loss of pyroglutamate moiety in product ion spectrum, as shown in FIG. 4B.

The formation of the adduct of diclofenac acylglucuronide with ECO which was detected in the present method is as outlined in Scheme E4, below.

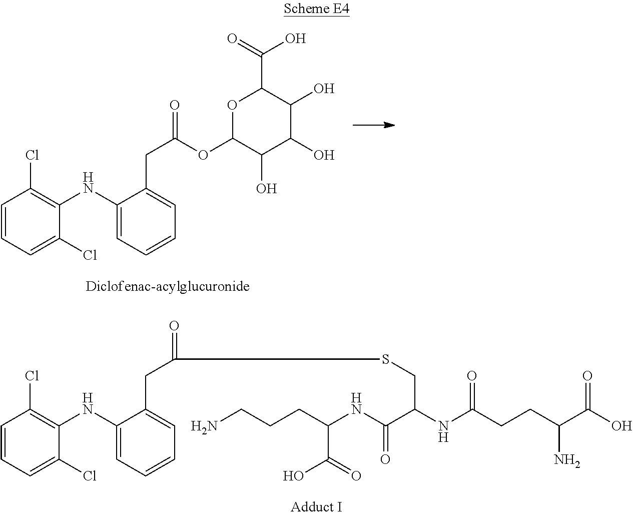

### Example 7-9

### Detecting Soft and/or Hard Reactive Metabolites

Following the procedure as described in Example 1 above, the method of the present invention and ECHK were applied to detect reactive metabolites of test compounds which are known to produce “soft” and/or “hard” reactive metabolites, using neutral loss scanning of 129 Da.

### Example 7

### Diclofenac Analyzed by ECHK

Diclofenac was selected as a test compound to evaluate the usefulness of the present method and ECHK to detect “soft” reactive metabolites.

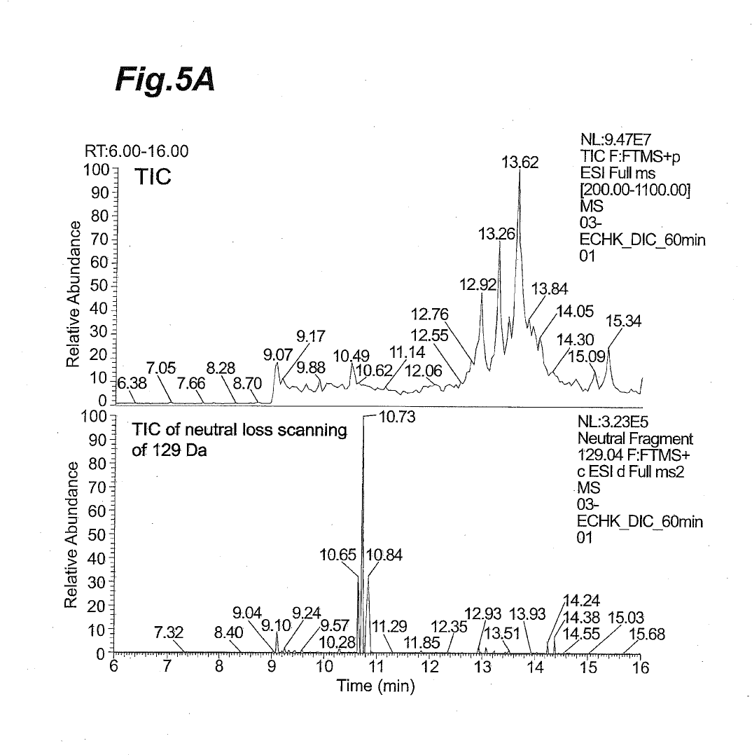

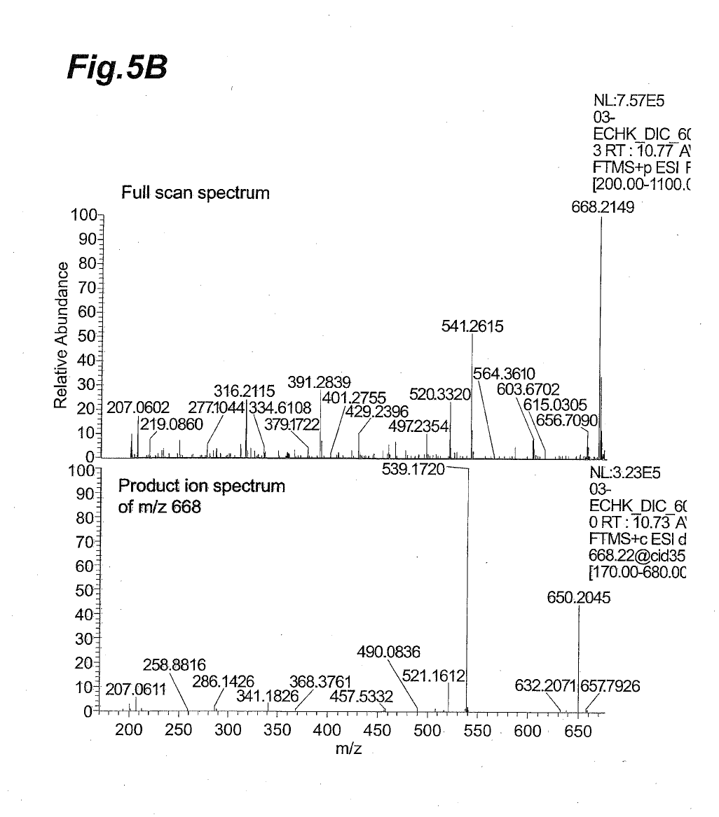

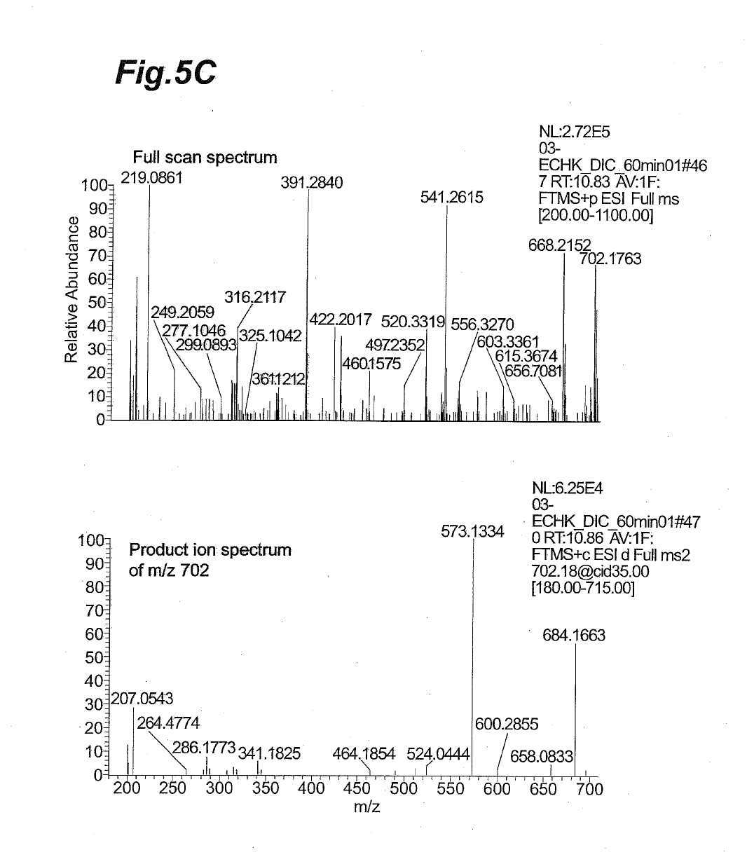

FIG. 5A shows TIC and TIC of neutral loss scanning of 129 Da obtained for the reaction mixture. The most intense peak which was detected in TIC of neutral loss scanning of 129 Da at retention time of 10.7 min included two adducts of reactive metabolites of diclofenac with ECHK which showed the protonated ions at m/z 668 and 702. Full scan and product ion spectra of the two adducts were shown in FIGS. 5B and 5C, respectively.

The formation of the two adducts of reactive metabolites of diclofenac with ECHK which were detected in the present method is as outlined in Scheme E5, below.

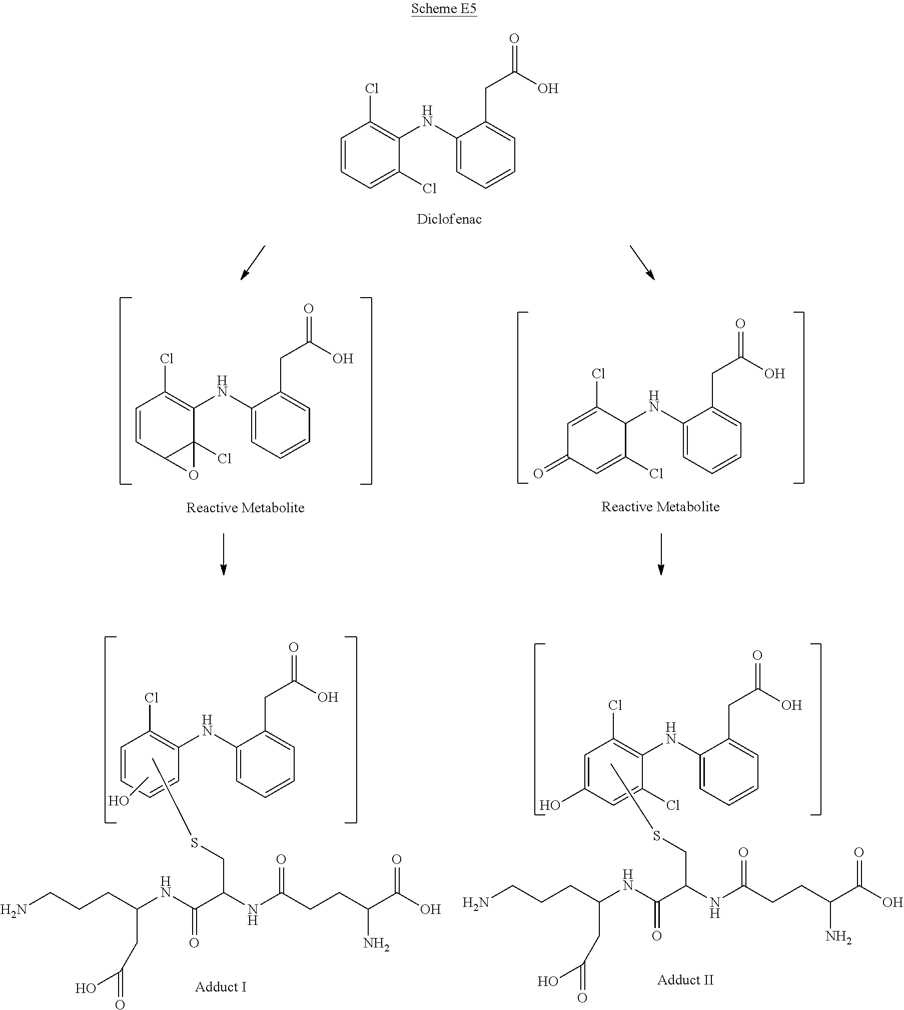

### Example 8

### Clozapine Analyzed by ECHK

Clozapine was selected as a test compound to evaluate the usefulness of the present method and ECHK to detect a “soft” reactive metabolite.

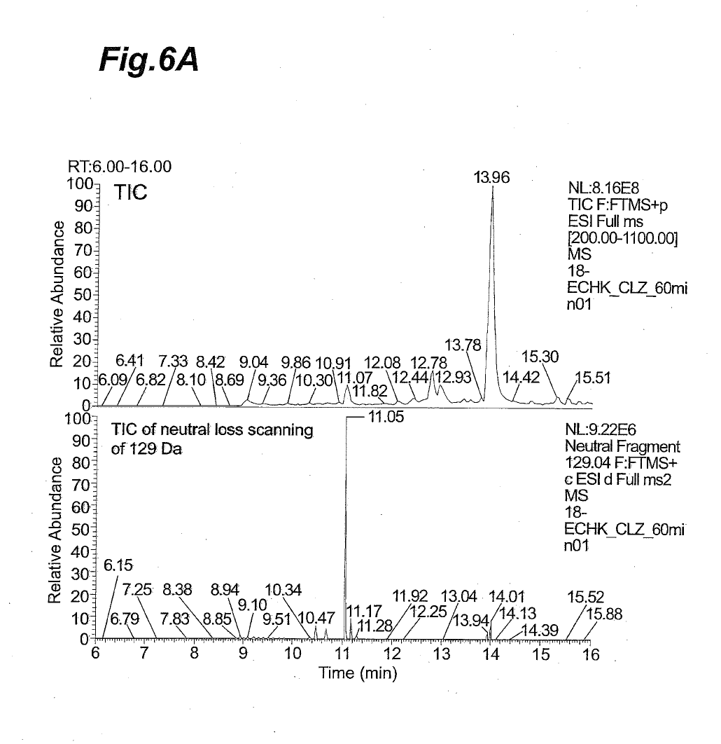

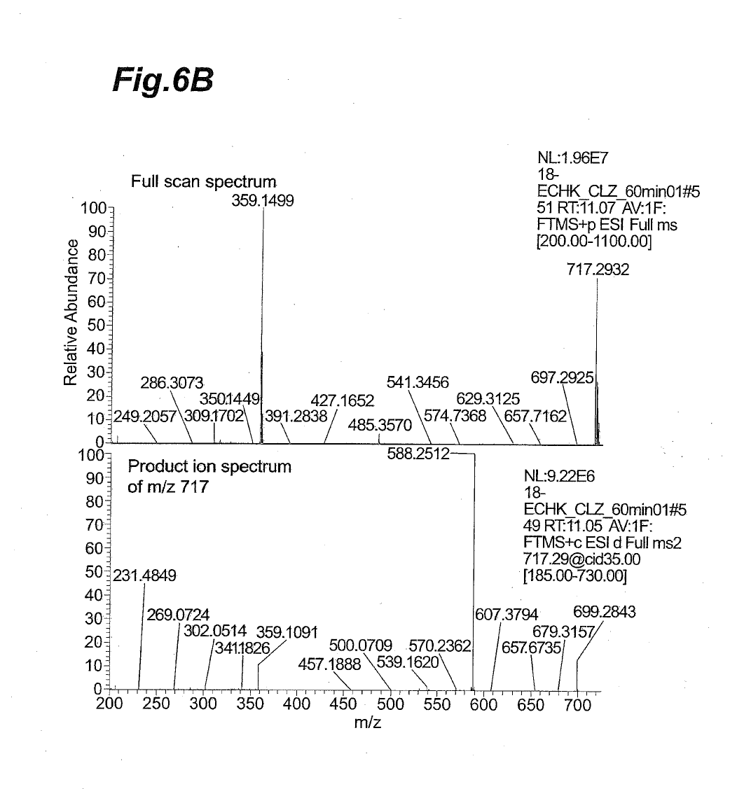

FIG. 6A shows TIC and TIC of neutral loss scanning of 129 Da obtained for the reaction mixture. The most intense peak which was detected in TIC of neutral loss scanning of 129 Da at retention time of 11.1 min included a single adduct which showed the protonated ion at m/z 717. Full scan and product ion spectra of this adduct was shown in FIG. 6B.

The formation of adduct of the reactive metabolite of clozapine with ECHK which was detected in the present method is as outlined in Scheme E6, below.

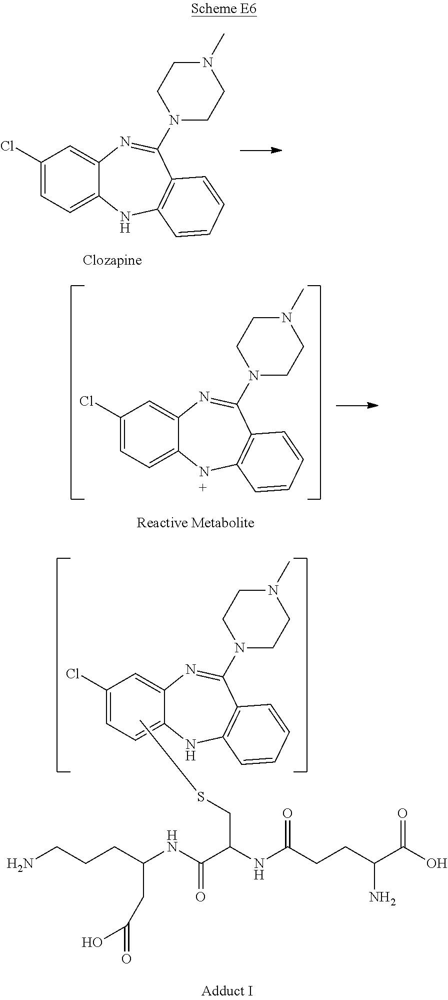

### Example 9

### 2-[2-Thienyl]-furan Analyzed by ECHK

2-[2-Thienyl]-furan was selected as a test compound to evaluate the usefulness of the present method and ECHK to detect both “soft” and “hard” reactive metabolites.

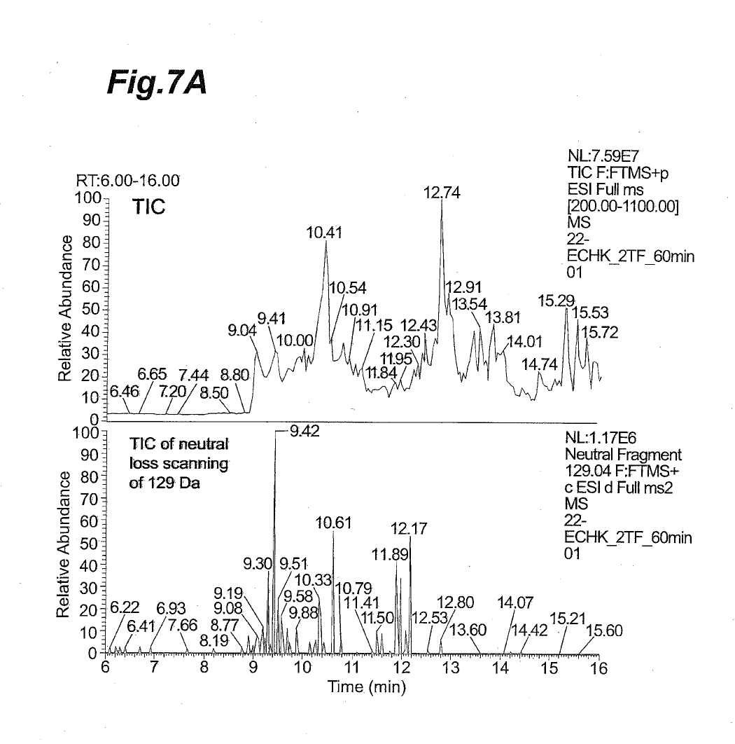

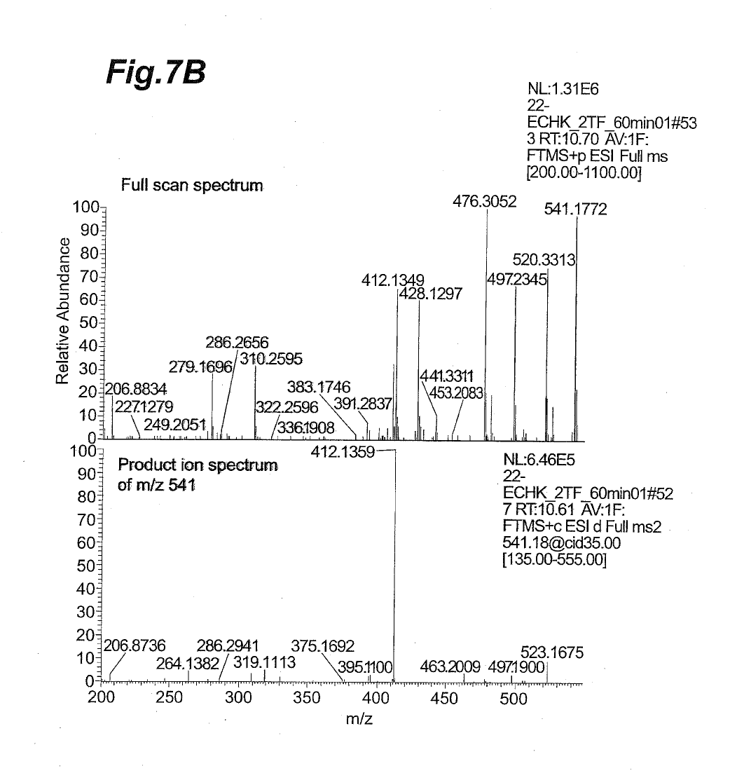

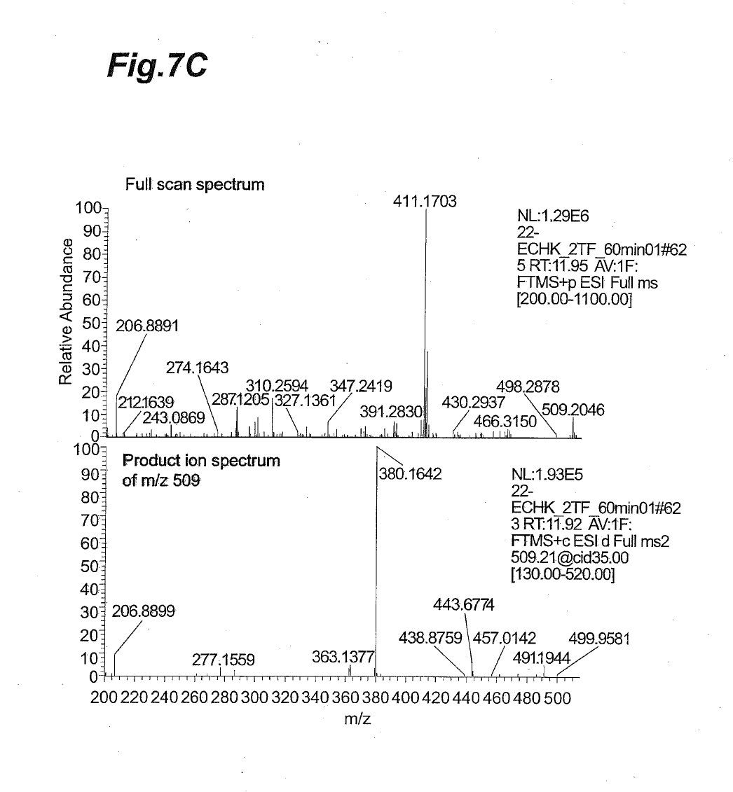

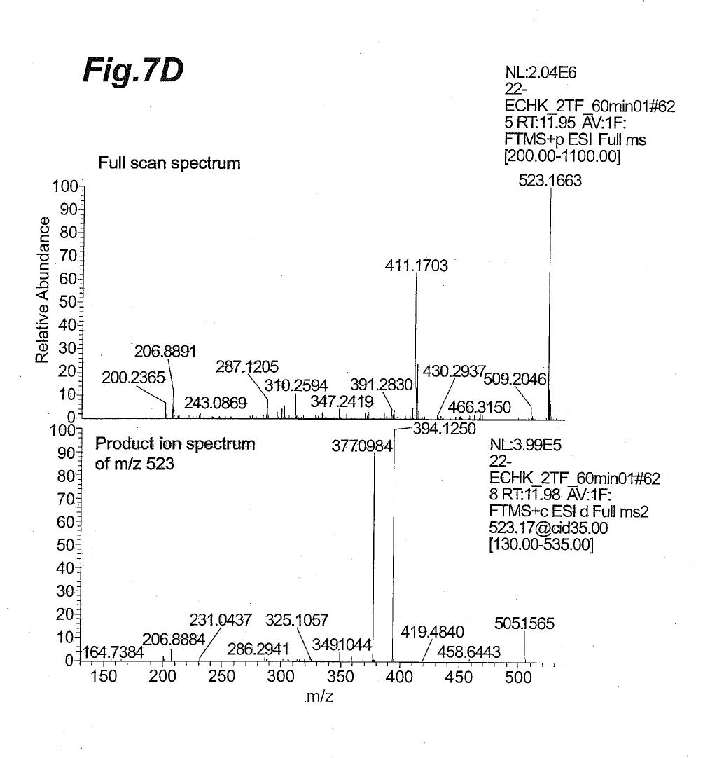

FIG. 7A shows TIC and TIC of neutral loss scanning of 129 Da obtained for the reaction mixture. Two intense peaks were detected in TIC of neutral loss scanning of 129 Da at retention time of 10.6 min and 11.9 min. The first component at 10.6 min showed the protonated ion at m/z 541 in full scan spectrum and the product ion at m/z 412 which indicated the loss of pyroglutamate moiety in product ion spectrum, as shown in FIG. 7B. The second component at 11.9 min included two adducts of reactive metabolites of 2-[2-Thienyl]-furan with ECHK and showed the protonated ions at m/z 509 and 523 in full scan spectrum and the product ions at m/z 380 and 394 which indicated the loss of pyroglutamate moiety in product ion spectrum, as shown in FIGS. 7C and 7D.

The formation of the three adducts of reactive metabolites of 2-[2-thienyl]-furan with ECHK which were detected in the present method is as outlined in Scheme E7, below.

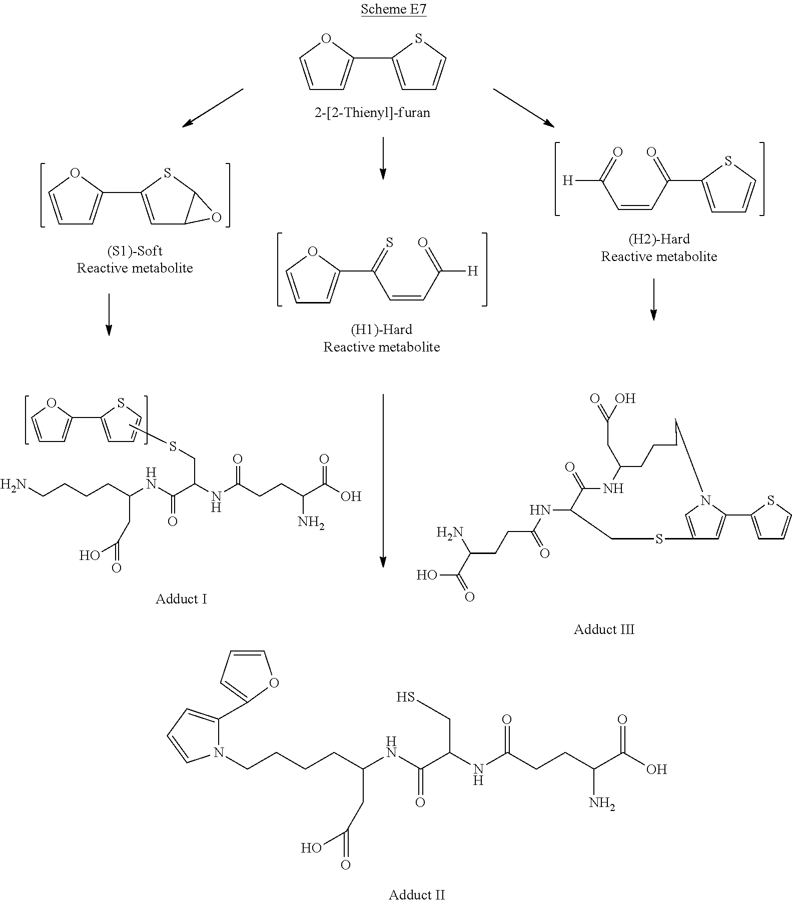

### Example 10

### Diclofenac Acylglucuronide Analyzed by ECHK

Following the procedure as described in Example 5, the method of the present invention and ECHK were applied to detect reactive metabolites which were produced by UDP-glucuronosyltransferase.

Diclofenac acylglucuronide was selected as a test compound to evaluate the usefulness of the present method and ECHK to detect reactive metabolites which were produced by UDP-glucuronosyltransferase.

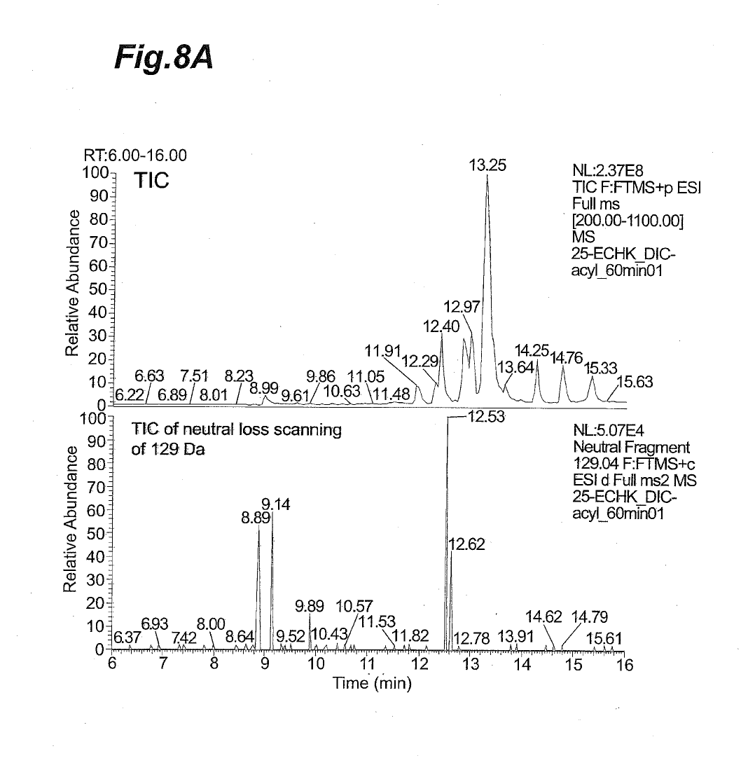

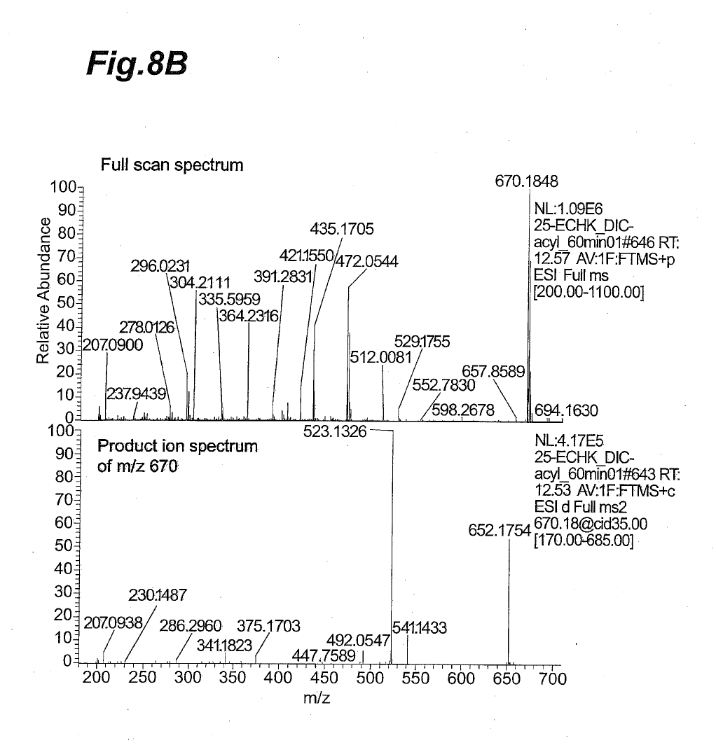

FIG. 8A shows TIC and TIC of neutral loss scanning of 129 Da obtained for the reaction mixture. An intense peak was detected in TIC of neutral loss scanning of 129 Da at retention time of 12.5 min. This peak showed the protonated ion at m/z 670 in full scan spectrum and the product ion at m/z 541 which indicated the loss of pyroglutamate moiety in product ion spectrum, as shown in FIG. 8B.

The formation of the adduct of diclofenac acylglucuronide with ECHK which was detected in the present method is as outlined in Scheme E8, below.

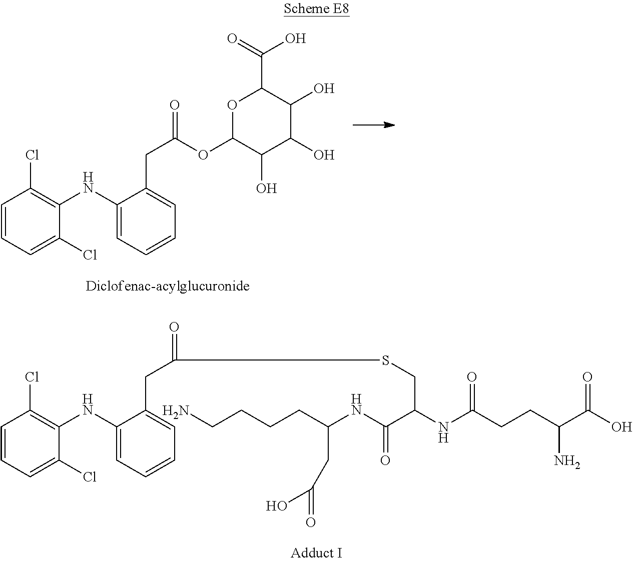

### Example 11-13

### Detecting Soft and/or Hard Reactive Metabolites

Following the procedure as described in Example 1 above, the method of the present invention and EHCK were applied to detect reactive metabolites of test compounds which are known to produce “soft” and/or “hard” reactive metabolites, using neutral loss scanning of 129 Da.

### Example 11

### Diclofenac Analyzed by EHCK

Diclofenac was selected as a test compound to evaluate the usefulness of the present method and PUCK to detect “soft” reactive metabolites.

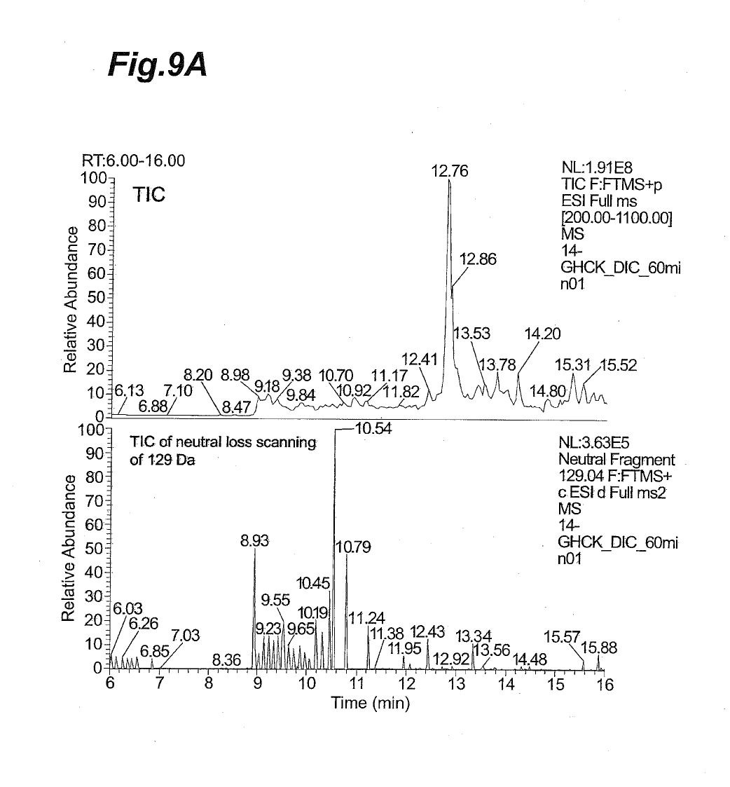

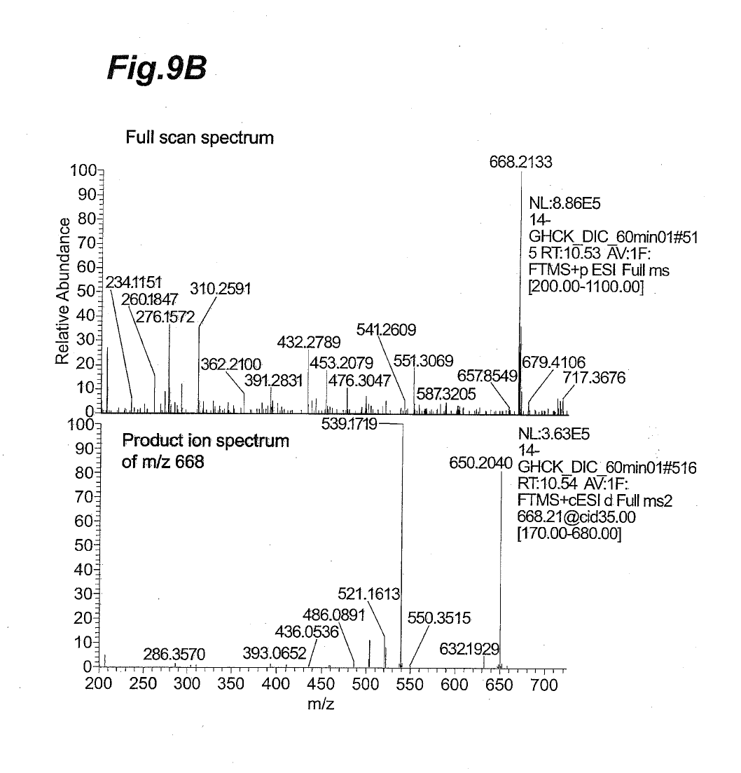

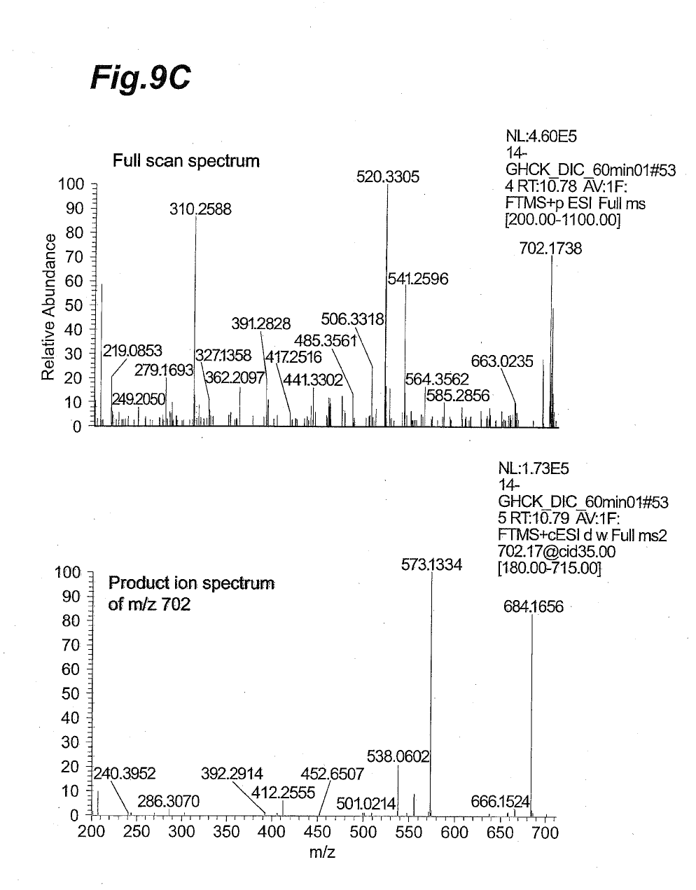

FIG. 9A shows TIC and TIC of neutral loss scanning of 129 Da obtained for the reaction mixture. Two intense peaks were detected in TIC of neutral loss scanning of 129 Da at retention time of 10.5 min and 10.8 min. The two components at 10.5 min and 10.8 min showed the protonated ions at m/z 668 and 702. Full scan and product ion spectra of the two adducts were shown in FIGS. 9B and 9C, respectively.

The formation of the two adducts of reactive metabolites of diclofenac with ECHK which were detected in the present method is as outlined in Scheme E9, below.

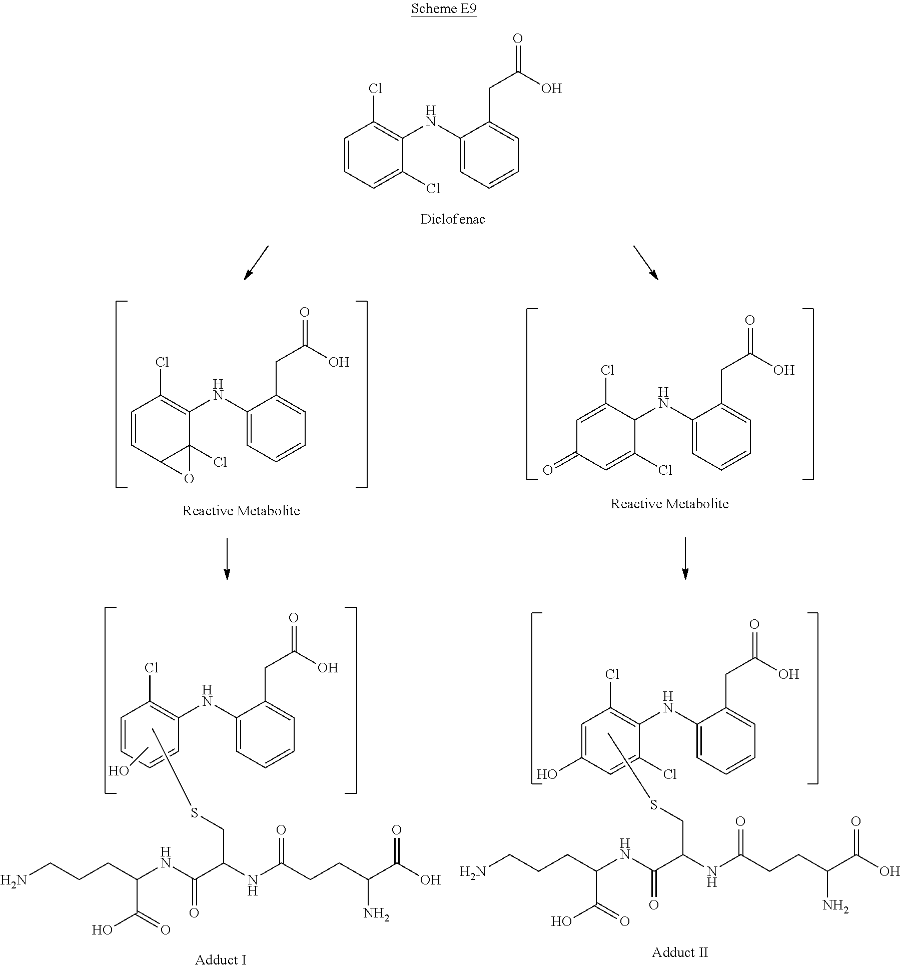

### Example 12

### Clozapine Analyzed by EHCK

Clozapine was selected as a test compound to evaluate the usefulness of the present method and EHCK to detect a “soft” reactive metabolite.

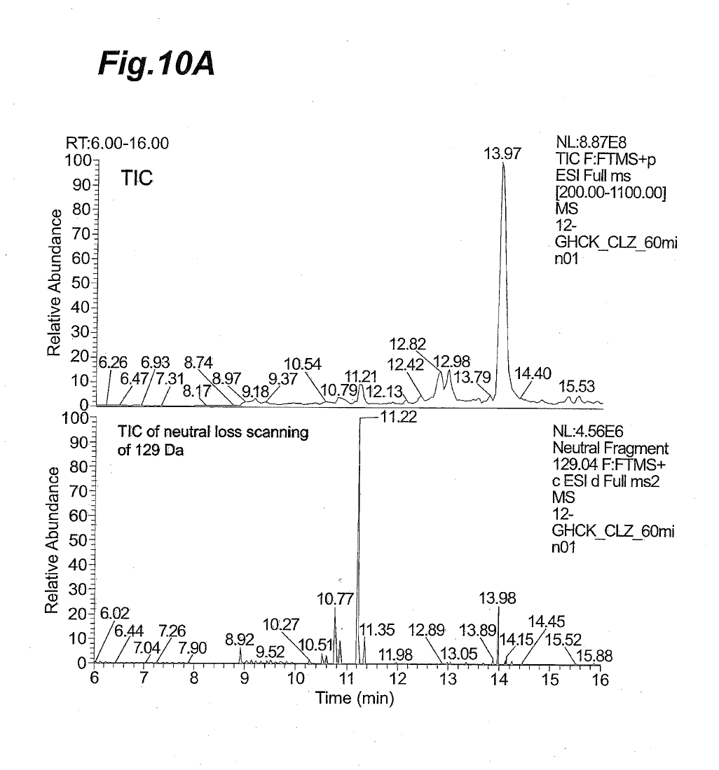

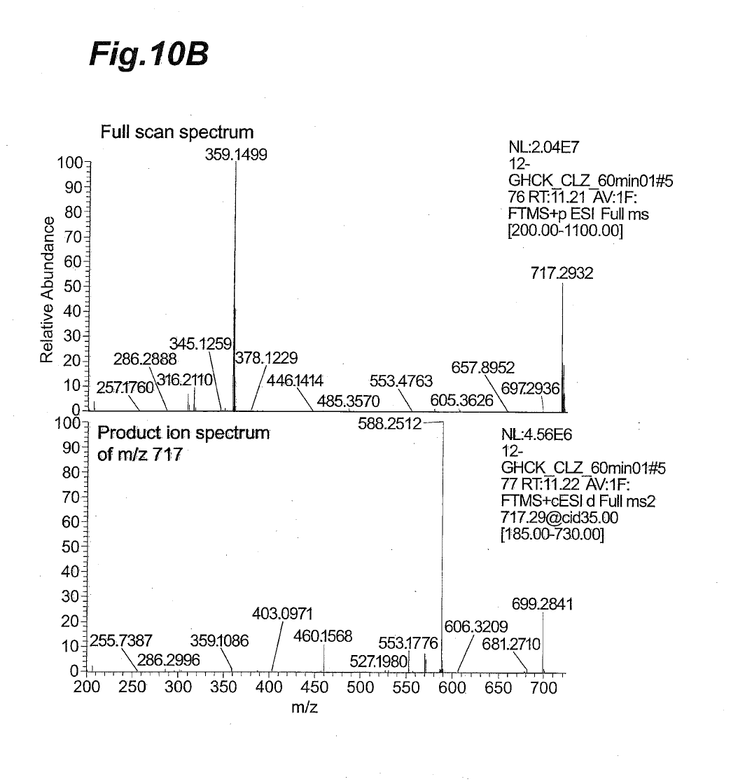

FIG. 10A shows TIC and TIC of neutral loss scanning of 129 Da obtained for the reaction mixture. The most intense peak which was detected in TIC of neutral loss scanning of 129 Da at retention time of 11.2 min included a single adduct which showed the protonated ion at m/z 717. Full scan and product ion spectra of this adduct was shown in FIG. 10B.

The formation of adduct of the reactive metabolite of clozapine with EHCK which was detected in the present method is as outlined in Scheme E10, below.

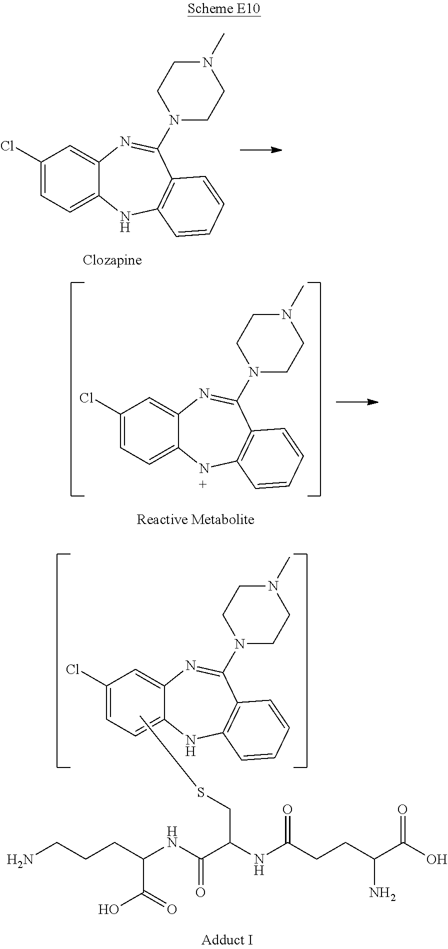

### Example 13

### 2-[2-Thienyl]-furan Analyzed by EHCK

2-[2-Thienyl]-furan was selected as a test compound to evaluate the usefulness of the present method and EHCK to detect both “soft” and “hard” reactive metabolites.

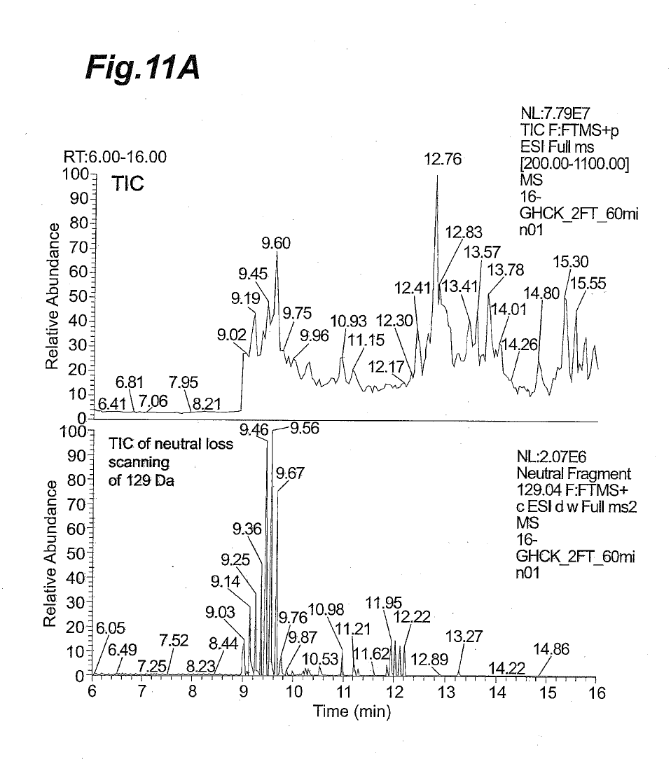

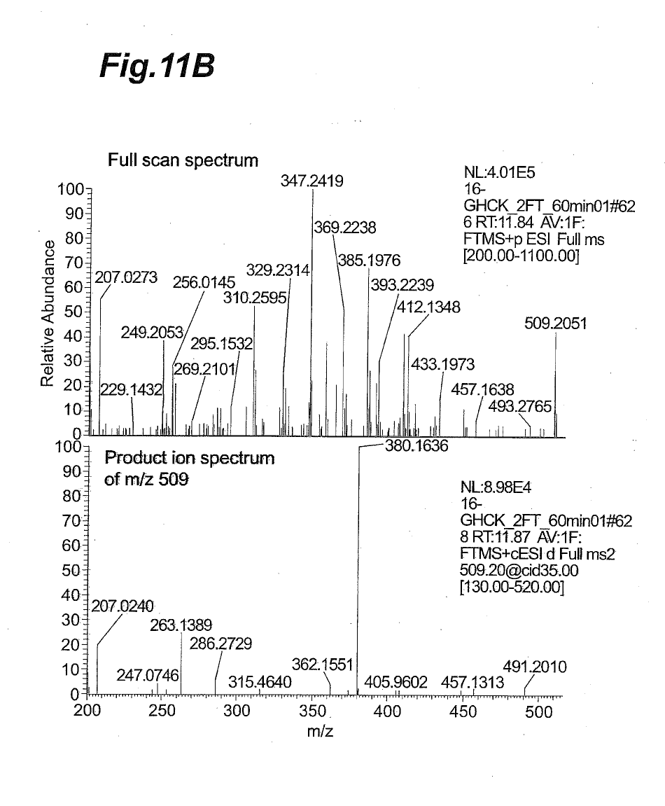

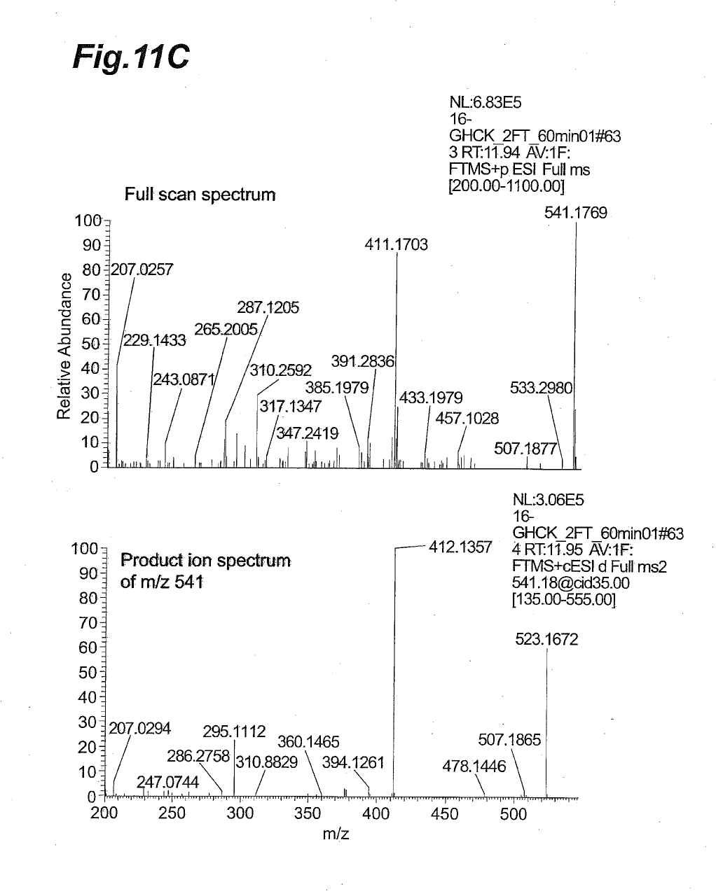

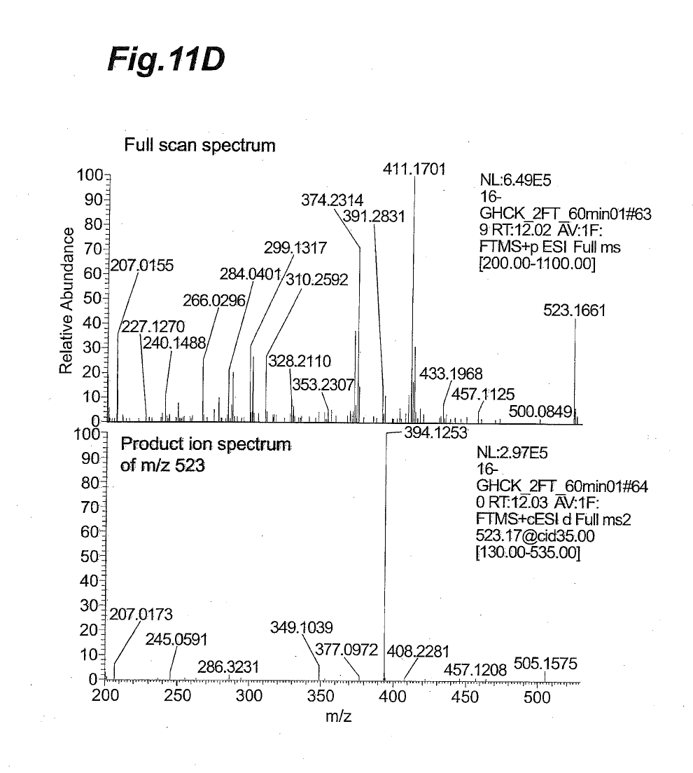

FIG. 11A shows TIC and TIC of neutral loss scanning of 129 Da obtained for the reaction mixture. The peaks which were detected in TIC of neutral loss scanning of 129 Da at retention time of 12.0 min included three adducts of reactive metabolites of 2-[2-Thienyl]-furan with EHCK which showed the protonated ions m/z 509, 541 and 523. Full scan and product ion spectra of the three adducts were shown in FIGS. 11B, 11C and 11D, respectively.

The formation of the three adducts of reactive metabolites of 2-[2-thienyl]-furan with EHCK which were detected in the present method is as outlined in Scheme E11, below.

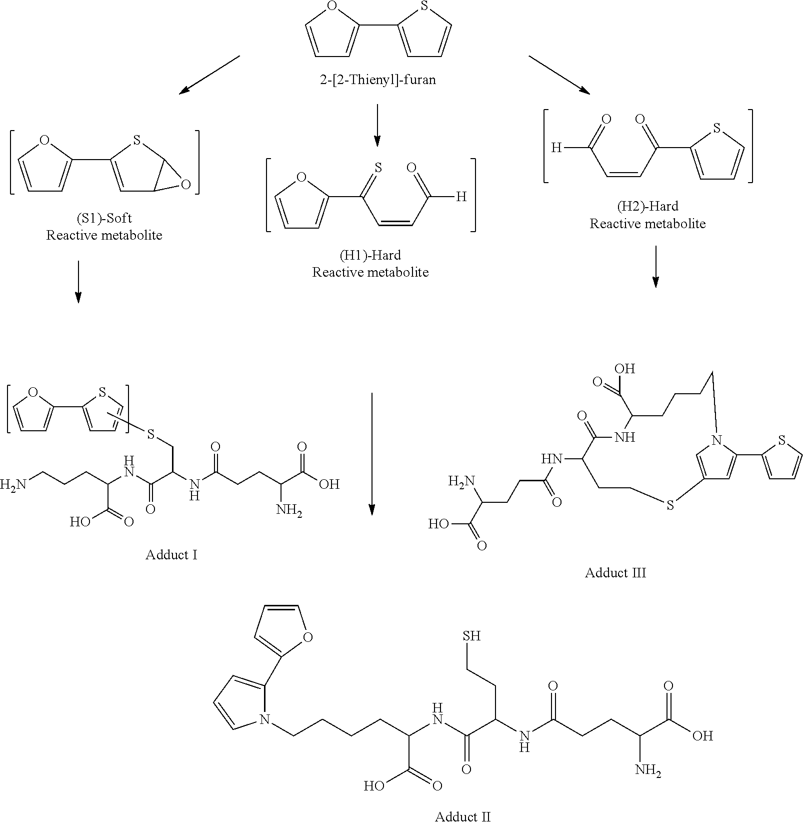

### Example 14

### Diclofenac Acylglucuronide Analyzed by EHCK

Following the procedure as described in Example 5, the method of the present invention and EHCK were applied to detect reactive metabolites which were produced by UDP-glucuronosyltransferase.

Diclofenac acylglucuronide was selected as a test compound to evaluate the usefulness of the present method and EHCK to detect reactive metabolites which were produced by UDP-glucuronosyltransferase.

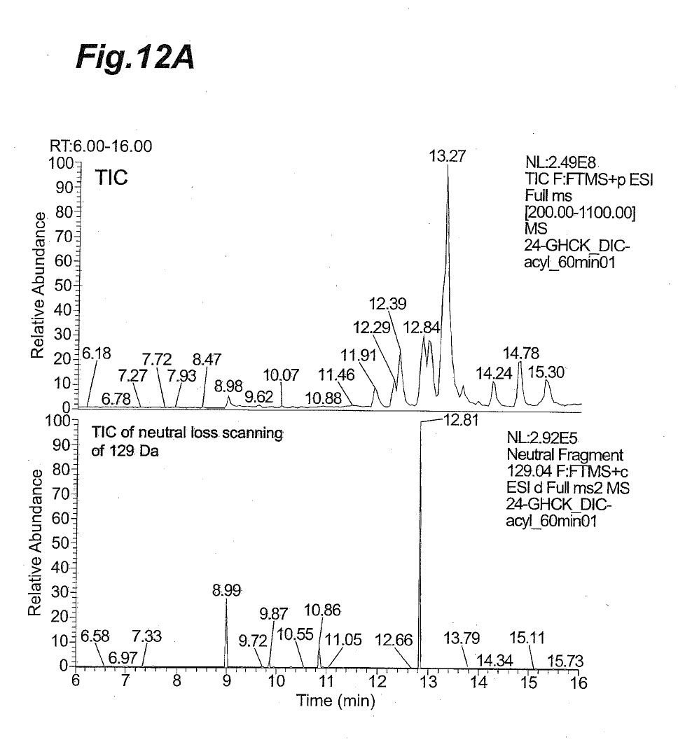

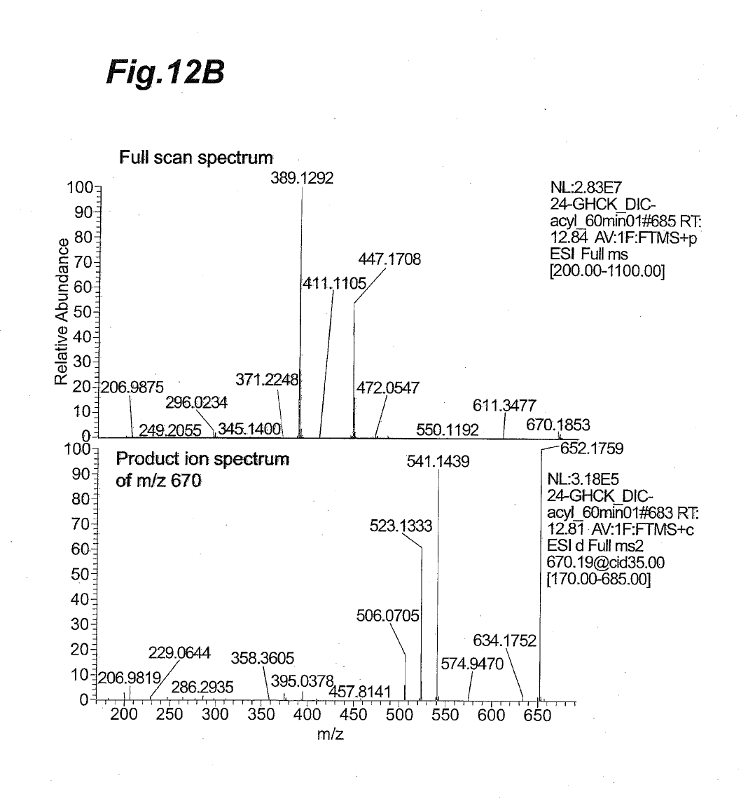

FIG. 12A shows TIC and TIC of neutral loss scanning of 129 Da obtained for the reaction mixture. An intense peak was detected in TIC of neutral loss scanning of 129 Da at retention time of 12.8 min. This peak showed the protonated ion at m/z 670 in full scan spectrum and the product ion at m/z 541 which indicated the loss of pyroglutamate moiety in product ion spectrum, as shown in FIG. 12B.

The formation of the adduct of diclofenac acylglucuronide with EHCK which was detected in the present method is as outlined in Scheme E12, below.

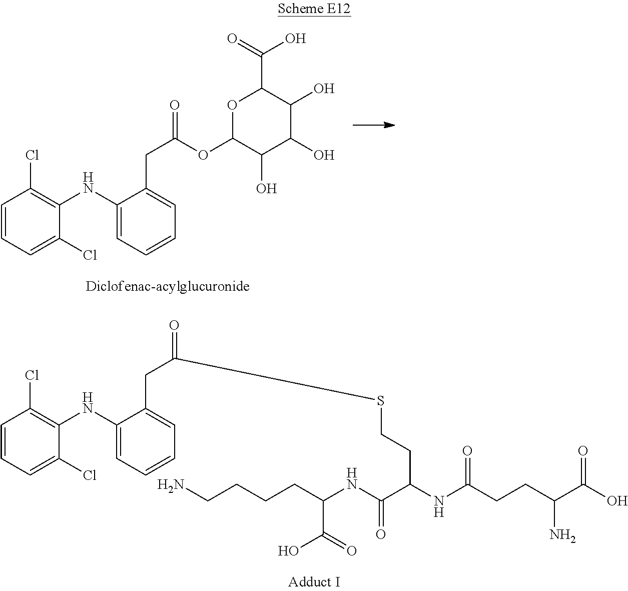

1. A compound of formula (I): 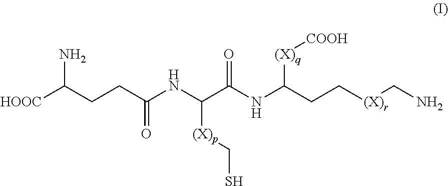
wherein
X is CH₂, and
p, q and r are each independently 0 or 1
with a proviso that the compound wherein p is 0, q is 0 and r is 1 is excluded.

2. The compound of claim 1, which is a compound of formula (II):

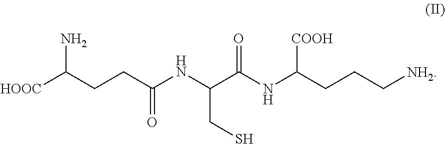

3. The compound of claim 1, which is a compound of formula (III):

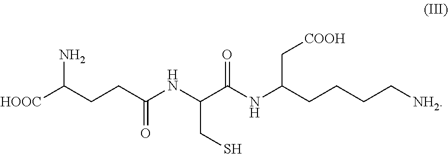

4. The compound of claim 1, which is a compound of formula (IV):

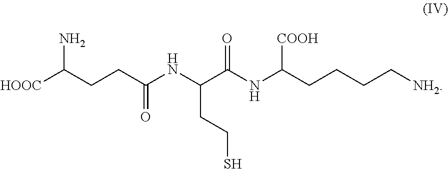

5. A method for detecting reactive metabolites of a test compound comprising
(a) incubating a test compound with a compound of formula (I): 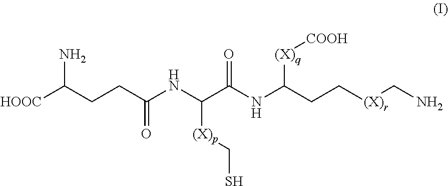
wherein
X is CH₂, and
p, q and r are each independently 0 or 1
with a proviso that the compound wherein p is 0, q is 0 and r is 1 is excluded,
and a drug metabolizing enzyme; to yield a product mixture comprising one or more adducts; and
(b) detecting the adducts of Step (a).

6. The method of claim 5, wherein the compound of formula (I) is a compound of formula (II): 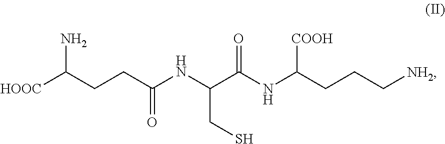
a compound of formula (III): 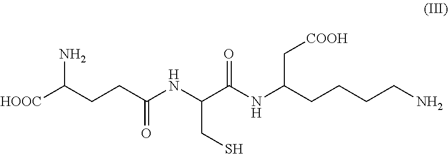
or a compound of formula (IV):

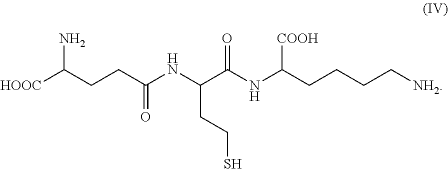

7. The method of claim 5, wherein the drug metabolizing enzyme is human, rat, mouse, hamster, dog, monkey or rabbit liver microsomes.

8. The method of claim 5, wherein the drug metabolizing enzyme is cytochrome P450 enzymes in combination with NADPH co-factor or cytochrome P450 enzymes in combination with NADPH regenerating system.

9. The method of claim 5, wherein the drug metabolizing enzyme is UDP-glucuronosyltransferases in combination with uridine diphosphoglucuronic acid (UDPGA) co-factor.

10. The method of claim 5, wherein the adducts are detected in a neutral loss mass spectrum.

11. The method of claim 10, wherein the neutral loss mass spectrum is measured using ESI-MS/MS or LS/MS.
